(19) United States
(12) Patent Application Publication
(10) Pub. No.: US 2008/0009748 A1
(43) Pub. Date: Jan. 10, 2008

(54) METHOD AND APPARATUS FOR THE DETERMINATION OF INTRINSIC SPECTROSCOPIC TUMOR MARKERS BY BROADBAND-FREQUENCY DOMAIN TECHNOLOGY

(75) Inventors: Enrico Gratton, San Clemente, CA (US); Shwayta Kukreti, Irvine, CA (US); Albert E. Cerussi, Lake Forest, CA (US); Bruce J. Tromburg, Irvine, CA (US)

Correspondence Address: MYERS DAWES ANDRAS & SHERMAN, LLP, 19900 MACARTHUR BLVD.,, SUITE 1150, IRVINE, CA, 92612, US

(73) Assignee: The Regents of the University of California, Okland, CA (US)

(21) Appl. No.: 11/749,704

(22) Filed: May 16, 2007

Related U.S. Application Data
(60) Provisional application No. 60/747,384, filed on May 16, 2006.

(51) Int. Cl. A61B 6/00

(52) U.S. Cl. 600/475; 600/473

(57) ABSTRACT

The illustrated embodiment is an improvement in a method of optically analyzing tissue in vivo in an individual to obtain a unique spectrum for the tissue of the individual, the improvement including the steps of optically measuring the tissue of the individual to obtain a spectrum of an optical parameter, and identifying a spectral signature specific to a metabolic or physiologic state in the tissue of the individual with a unique spectrum for the tissue by considering only the spectral differences between a first metabolic or physiologic state of the tissue of the individual and one or more other metabolic or physiologic states of the tissue of the individual such that identification of the spectral signature is self-referencing with respect to intra-individual metabolic or physiologic variations. The method also includes separating benign and malignant lesions only using the shape or a characteristic of the spectrum.

### RELATED APPLICATIONS

The present application is related to U.S. Provisional Patent Application Ser. No. 60/747,384, filed on May 16, 2006, which is incorporated herein by reference and to which priority is claimed pursuant to 35 USC 119.

### GOVERNMENT RIGHTS

This invention was made with Government support under Grant Nos. CA105480 and EB000559, awarded by the National Institutes of Health. The Government has certain rights in this invention.

### BACKGROUND OF THE INVENTION

1. Field of the Invention

The invention relates to the field of methods of use of near-IR for the determination of optical parameters of tissues and apparatus for performing the same.

2. Description of the Prior Art

Despite years of research, the promise of non-invasive optical biopsy of breast tumors has not been fully realized. During the past decade we witnessed a renewed interest in this field due to the realization that quantitative spectroscopy can be performed in thick tissues. The challenge of spectroscopy in tissues has been the separation of attenuation due to scattering from that due to tissue absorption. Methods to achieve this separation have been proposed based on the measurement of the time of flight of light pulses through the tissue. Most of the proposed methods measure the optical parameters at few selected wavelengths. It was believed that once quantification was achieved, the classification of tissues functional properties according to the recovered absorption and scattering parameters could be sufficient to distinguish tumors from normal tissue. Several investigators developed apparatuses and algorithms to quantify the amount of major tissue components, namely oxy and deoxyhemoglobin, water, lipids and the spectral dependence of scattering. Although the classification of tumors based on the relative amount of those basic tissue components showed clear correlation with some type of tumors, the sensitivity and specificity of this kind of analysis was not higher than 75-85%, depending on the method used and the kind of tumor.

It was also believed that three dimensional reconstruction of the tissue optical parameters will increase the contrast ratio to the point that the differences in optical parameters from one location to the other could differentiate the diseased tissue from the normal. A different approach that appeared very promising was to obtain detailed spectral information using a spectral continuum in the near-IR. This method has provided perhaps the best specificity in regard to separating normal from diseased tissue for breast cancer diagnosis.

The use of near-IR for the determination of optical parameters of tissues is a well established field with at least 50 years of research. The problem with determining the absorption in tissues is that the apparent absorption depends on the amount of scattering in the tissue. About 15 years ago it was suggested that by measuring the time of transit of a light pulse through the tissue it was possible to independently determine the amount of scattering. This principle was implemented in several embodiments, one of which is the described by U.S. patent application entitled “Quantitative broadband absorption and scattering spectroscopy in turbid media by combined Frequency-Domain and Steady State Methodologies”, Ser. No. 10/191,693, incorporated herein by reference.

Using time of transit of a light pulse through the tissue, a new wave of instruments was built with the purpose of identifying the major components of tissues. These components were the amount of water, lipid oxy and deoxyhemoglobin. To use the time of flight approach or the frequency-domain equivalent, called the phase shift approach, the light source needs to be pulsed or modulated at very high frequencies, typically in the 100 MHz range. None of these approaches can give the detailed wavelength information that is necessary for the applications of the methods described in this disclosure.

About 10 years ago, the group headed by Dr. Tromberg at UCI proposed to use a broadband approach to obtain detailed information about the spectra of the tissue. Although the method of using a range of wavelength was described and how to combine the scattering information obtained with the frequency domain approach was also implemented, the analysis of the data was carried out based on the idea of recovering information regarding the amount of the tissue components from the detailed spectra. None of the work done neither at UCI nor in other laboratories was done with the purpose of identifying specific tumor spectral components.

Within the field of optical mammography, tumor tissue is separated from normal tissue in the following manner: Tissues are classified based on the relative amounts of the major tissue components, namely, (oxyhemoglobin, deoxyhemoglobin, water and lipid). The major breast tissue components are quantified by fitting the spectroscopic absorption data with pre-assigned spectra or principal component analysis. However, the knowledge of the amount of tissue components is not enough to uniquely identify tumors, even less to distinguish between malignant and benign tumors. The disadvantage of the available method is that the sensitivity is not 100%. Of course, the gold standard for breast tumor screening is mammography. However, x-ray mammography cannot be applied in young women due to the dense spectroscopic breast from the x-ray point of view.

Furthermore, mammography has limited sensitivity and specificity. Mammography is also painful and the results must be read by an experienced radiologist. No automatic method for the identification of tumors from the mammography slide is available.

Optical methods offer a non-invasive view to molecular compositional and functional changes in tissue. Diffuse optical spectroscopy (DOS) and diffuse optical imaging (DOI) methods have shown to be sensitive to changes in tumor angiogenesis and hypoxia in breast tissue by measuring tissue hemoglobin concentration and oxygen saturation. In these approaches the measured absorption spectra are usually obtained at discrete wavelengths between 650-850 nm and then translated to obtain concentrations of oxyhemoglobin and deoxyhemoglobin by fitting to hemoglobin extinction spectra. Several groups have increased spectral resolution by including more wavelengths thereby obtaining amounts of bulk lipid and water. Recently, a study of 58 malignant tumors revealed that deoxyhemoglobin, bulk lipid and water levels are significantly different for tumors comparison to normal tissue. Alterations in these parameters are correlated to local structural and functional changes in breast physiology during cancer; they are not unique to cancer as the same components are also found in normal tissue.

### BRIEF SUMMARY OF THE INVENTION

The illustrated embodiment of the invention includes the step of combining a new analysis method with the data obtained with the instrument described in patent application Ser. No. 10/191,693, which is incorporated herein by reference. We have been able to identity spectral signatures that are specific to tumors. The described instrument is needed for the correction of the spectral data for the scattering contribution.

In the method of the illustrated embodiment we consider only the spectral differences between normal and diseased tissue. Note that we proceed by first subtracting the absorption spectrum of the normal breast from spectra obtained at different locations on the breast with the tumor. We then fit this difference spectra using the basis components spectra, and then analyze the residuals of this fit. Note that for this work it is important that we have a complete absorption spectrum of the tissue across the full near-IR wavelength range of 650-1000 nm.

Thus as stated above, for this we use the broadband approach as described in patent application Ser. No. 10/191,693 to separate scattering and obtain an absolute absorption spectra. Furthermore, in order to use our analysis method, we use the data from instrument described in the patent application Ser. No. 10/191,693.

We have developed a double differential method to analyze the near infrared spectra of regions of the breast with tumors. As stated above we consider only the spectral differences between normal and diseased tissue by fitting the differential spectra using the basis components spectra and then analyzing the residuals of this fit. This differential approach can be performed by comparison between regions of normal and tumor breast tissue. With this method we show intrinsic spectroscopic markers of breast tumors in the near-IR. We show that using the double differential method, the near-IR spectra of regions of the breast with tumors reveals characteristic absorption bands in the lipid region that were previously unnoticed.

Furthermore, the water band in the 980 nm region also shows distinct variations in the tumor region compared to the normal breast. By combining this information, we constructed an index that is characteristic of the tumor region (100% specificity and sensitivity for the 12 patients investigated) and has the potential to distinguish tumors on the basis of the on the basis of the tissue composition and/or molecular disposition of tissue components.

The proposed optical method can be applied to women of all ages, is not painful and the result of the analysis can be interpreted by a computer algorithm. The instrumentation does not produce harmful radiation and it can be installed in a doctor's cabinet. The instrumentation is on order of magnitude less expensive that the conventional mammography method.

The analysis method of the illustrated embodiment can be used for the early diagnosis of tumors. The invention is not limited to breast cancer, but it could be applied to other type of tumors, for example for prostate cancer. In principle, the method described in this disclosure could completely replace conventional mammography.

The double differential method and spectral separation method of the illustrated embodiment can be used for identification and characterization of changes in an individual patient's tissue metabolic and physiologic states. These include but are not limited to:

1) appearance, progression, and regression of diseases such as cancer

2) distinguishing between malignant and benign disease processes

3) determining the response of an individual to therapies for disease prevention (e.g chemoprevention), reversal (e.g. chemotherapy), and long term clinical management to treat benign conditions or cancer risk (e.g. hormonal and SERM (synthetic estrogen receptor modulators) therapies.

In an effort to improve the sensitivity and specificity from optical methods we use the double-differential approach to spectral analysis of near-IR (650-1000 nm) absorption spectra to explore spectroscopic absorption signatures. Briefly, this is a self-referencing method which accounts for individual physiological variation. Furthermore the method accounts for concentration differences between tumor and normal tissue due to the major near-infrared breast tissue absorbers (oxyhemoglobin, deoxyhemoglobin, bulk lipid and water) to reveal unique markers of the tumor.

The method of the invention can be equivalently applied to any tissue. Here by way of example only we apply the double-differential approach to breast tissue absorption spectra to discriminate benign and malignant lesions, a challenging problem for near-infrared. This is also a retrospective study to evaluate if DOS can discriminate benign and malignant lesions. More specifically, the question we address is: are there spectral differences between benign and malignant lesions?

More particularly, the illustrated embodiments of the invention include an improvement in a method of optically analyzing tissue in vivo in an individual to obtain a unique spectrum for the tissue of the individual. The improvement comprises the steps of optically measuring the tissue of the individual to obtain a spectrum of an optical parameter, and identifying a spectral signature specific to a metabolic or physiologic state in the tissue of the individual with a unique spectrum for the tissue by considering only the spectral differences between a first metabolic or physiologic state of the tissue of the individual and one or more other metabolic or physiologic states of the tissue of the individual such that identification of the spectral signature is self-referencing with respect to intra-individual metabolic or physiologic variations.

The step of identifying the spectral signature specific to the metabolic or physiologic state in the tissue of the individual comprises the steps of subtracting the absorption spectrum of the first metabolic or physiologic state of the tissue from absorption spectrum obtained at different locations on tissue having at least one of the other metabolic or physiologic states to obtain a difference spectrum, fitting the difference spectrum to spectral basis components, and analyzing residuals of the spectral basis components from the fitted difference spectrum.

The step of identifying the spectral signature specific to the metabolic or physiologic state in the tissue of the individual also comprises obtaining a complete absorption spectrum of the tissue across the full IR, near-IR, or visible wavelength range.

The step identifying the spectral signature specific to the metabolic or physiologic state in the tissue of the individual may comprise the step of separating out a scattering spectrum and obtaining an absolute absorption spectrum.

The step of identifying the spectral signature specific to the metabolic or physiologic state in the tissue of the individual also comprises the steps of analyzing a near infrared spectrum of regions of a breast with a tumor by comparison between regions of normal breast tissue and tumor breast tissue by first subtracting the spectrum of the normal breast tissue of the individual from the spectrum obtained at different locations in the breast tissue with the tumor of the individual to obtain a differential spectrum, then fitting the differential spectrum using a basis component spectrum to obtain a fitted spectrum, and analyzing residues of the fitted spectrum.

The step of identifying the spectral signature specific to the metabolic or physiologic state in the tissue of the individual in one embodiment comprises the step of identifying intrinsic spectroscopic markers of the tissue in the near-IR. The step of identifying intrinsic spectroscopic markers of the tumor in the near-IR comprises step of identifying characteristic absorption bands indicative of state changes related to lipid, water, hemoglobin, derivatives of hemoglobin, or an optical absorber. The step identifying characteristic absorption bands in the lipid region comprises the step of characterizing variations in a water band in the 980 nm region in a tumor region of the individual compared to the normal tissue of the individual.

The step of identifying the spectral signature specific to the metabolic or physiologic state in the tissue of the individual in another embodiment comprises the step of combining information relating to spectral differences between tissue of the individual characterized by the first metabolic or physiologic state and tissue of the individual characterized by one or more other metabolic or physiologic states to construct an index that is characteristic of a region characterized by the one or more other metabolic or physiologic states on the basis of the lipid composition and/or bound water.

The step of identifying the spectral signature specific to the metabolic or physiologic state in the tissue of the individual in yet another embodiment comprises automatically identifying a spectral signature specific to the one or more other metabolic or physiologic states by a computer algorithmic procedure without physician intervention.

The step of identifying the spectral signature specific to a metabolic or physiologic state in the tissue of the individual with a unique spectrum for the tissue in an embodiment comprises the step of separating tissue having the first metabolic or physiologic state from tissue having the one or more other metabolic or physiologic states using only one or more characteristics of shape of the spectrum.

The step of separating tissue having the first metabolic or physiologic state from tissue having the one or more other metabolic or physiologic states using only one or more separation characteristics of shape of the spectrum in one embodiment comprises the step of separating benign and malignant lesions using only spectral shape.

The step of separating tissue having the first metabolic or physiologic state from tissue having the one or more other metabolic or physiologic states using only one or more separation characteristics of shape of the spectrum comprises using the concentration of hemoglobin or tissue optical index (TOI) value as a separation characteristic.

The step of separating tissue having the first metabolic or physiologic state from tissue having the one or more other metabolic or physiologic states using only one or more separation characteristics of shape of the spectrum in one embodiment comprises the step of discriminating more than two lesions types.

The step of separating tissue having the first metabolic or physiologic state from tissue having the one or more other metabolic or physiologic states using only one or more characteristics of shape of the spectrum in an embodiment comprises the step of determining a wavelength weighted distance of a given specific tissue component (STC) spectrum from a representative average spectrum of each metabolic or physiologic state of tissue. In the case where tumors are identified, the specific tissue component can be understood to be a specific tumor component.

The step of separating tissue having the first metabolic or physiologic state from tissue having the one or more other metabolic or physiologic states using only one or more characteristics of shape of the spectrum in another embodiment comprises the step of computationally determining spectral shape to discriminate between each metabolic or physiologic state of tissue.

The step of separating tissue having the first metabolic or physiologic state from tissue having the one or more other metabolic or physiologic states using only one or more characteristics of shape of the spectrum in yet another embodiment comprises the step of computationally determining spectral shape to discriminate between benign and malignant lesions.

The step of determining a wavelength weighted distance of a given spectrum from the representative average spectrum comprises the steps of determining a best set of weighting factors, storing the weighting factors, applying the stored weighting factors to a given a spectrum of unknown origin, and determining how distant the spectrum is from the average STC spectrum for each metabolic or physiologic state of tissue.

The step of optically measuring the tissue of the individual to obtain a spectrum of an optical parameter in various embodiments comprises obtaining the spectral signature as an absorption spectrum by combining frequency-domain and steady state measurements, by performing steady state measurements only, by performing time-domain measurements, or by performing spatially resolved measurements.

Another embodiment includes a software program recorded on a medium containing instructions for controlling a measurement and computer system for performing the steps of each of the improvements in the method above.

The illustrated embodiment also includes an apparatus for performing each of the embodiments of the improvements in the above method.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a graph of the basis spectra set used to describe the major tissue components in the breast.

FIG. 2 is a graph of the absorption spectra obtained at 11 locations along the line in the inset in the upper left portion of the graph, which is photograph of a frontal view of the breast being measured.

FIG. 3 is a graph of the spectra at the line location indicated in the inset of FIG. 2 which were subtracted by the average spectrum of the normal breast obtained at 11 locations in the symmetric position with respect to the breast with the tumor.

FIG. 4 is a graph of the spectral residuals corresponding to equation 5 below.

FIGS. 5a and 5b are graphs of the local residual variance at 11 positions in the breast with tumors along the line indicated in the inset of FIG. 2, for the tumor side and for the normal breast (Patient #30), respectively.

FIG. 6 is a graph of the correlation plot of the ratio of the standard deviation of region 1 verses the standard deviation of region 3. Each bar corresponds to a different patient. The bars on the left of the graph correspond to invasive carcinoma and bars on the right of the graph to fibro-adenoma patients. Region 1 corresponds to hemoglobin signals. A negative correlation means that the signal in region 1 of the STC component is negative on the average in that region.

FIG. 7 is a graph of the STC index verses the size of the tumor. There is no apparent correlation between the size of the tumor and the STC index.

FIG. 8 is a diagram of one apparatus in which the method of the illustrated embodiment of the invention may be practiced.

FIG. 9 is a graph of the amplitude of normalized STC spectra for 12 fibroadenoma lesions in the 24 lesions data set. The left vertical axis indicates the absorption units for the STC spectra and the horizontal axis is wavelength. The dotted “rectangular blocks” represent the wavelength regions weighted for “best discrimination” of cancer and fibroadenoma. Relative weightings shown on the right vertical axis.

FIG. 10 is a graph of the amplitude normalized STC spectra for 12 cancer from 24 lesions data set Amplitude normalized STC spectra for 12 cancer lesions in the 24 lesions data set. The left vertical axis indicates the absorption units for the STC spectra. The dotted “rectangular blocks” represent the wavelength regions weighted for “best discrimination” of cancer and fibroadenoma. Relative weightings shown on the right vertical axis.

FIG. 11 is a graph of the amplitude normalized STC spectra for 18 fibroadenoma from 40 lesions data set. The left vertical axis indicates the absorption units for the STC spectra. The dotted “rectangular blocks” represent the wavelength regions weighted for “best discrimination” of cancer and fibroadenoma. Relative weightings shown on the right vertical axis.

FIG. 12 is a graph of the amplitude normalized STC spectra for 22 fibroadenoma lesions in the 40 lesions data set. The left vertical axis indicates the absorption units for the STC spectra. The dotted “rectangular blocks” represent the wavelength regions weighted for “best discrimination” of cancer and fibroadenoma. Relative weightings shown on the right vertical axis.

FIG. 13 is a plot or the separation of 12 fibroadenoma and 12 cancer patients using equal weightings of the wavelength regions of the STC spectra. D_{c} and D_{f} refer to the “distance” or the “discrimination” from the average STC spectra for cancer and fibroadenoma, respectively. The “score” is the score calculated from algorithm for best weighting using equations as described below. The plot shows fibroadenoma plotted as empty triangles, cancer plotted as solid circles, and reference points plotted as solid squares.

FIGS. 14a-14d are separation plots for 24 lesions with unequal wavelength weighting, namely separation of 12 fibroadenoma and 12 cancer patients using unequal wavelength weightings of the STC spectra. FIG. 14a shows the weightings using 40 wavelength points (denoted as p=40). FIGS. 14b-14d show the weightings using p=20, 10 and 5, respectively. D_{c} and D_{f} refer to the “distance” or the “discrimination” from the average STC spectra for cancer and fibroadenoma, respectively. The “score” is the score calculated from algorithm for best weighting using equations in the disclosure. The plot shows fibroadenoma plotted as empty triangles, cancer plotted as solid circles, and reference points plotted as solid squares.

FIG. 15 is a separation plot for 24 lesions-determination of sensitivity and specificity. Separation of 12 fibroadenoma and 12 cancer patients using unequal wavelength weightings of the STC spectra with p=20. D_{c} and D_{f} refer to the “distance” or the “discrimination” from the average STC spectra for cancer and fibroadenoma, respectively. The “score” is the score calculated from algorithm for best weighting using equations as described in the specification. The plot shows fibroadenoma plotted as empty triangles, cancer plotted as solid circles, and reference points plotted as solid squares. Depending on the way the line of separation is drawn, lesions can be discriminated with 100% sensitivity and 92% specificity (Line 1) or 92% sensitivity and 100% specificity (Line 2).

FIGS. 16a and 16b are separation plots for 40 lesions-by locations averaged. The Figures show separation of 18 fibroadenoma and 22 cancer patients using weightings with p=5 wavelength points of the STC spectra. D_{c} and D_{f} refer to the “distance” or the “discrimination” from the average STC spectra for cancer and fibroadenoma, respectively. The “score” is the score calculated from algorithm for best weighting using equations as described below. The plot shows fibroadenoma plotted as empty triangles, cancer plotted as solid circles, and reference points plotted as solid squares. FIG. 16a shows the separation by averaging all positions of “line scan,” which refers to a line of points at which data was obtained. This “line scan” extends from normal tissue on one side of the lesion, over the lesion, and normal tissue on the other side of the lesion. FIG. 16b shows the separation by averaging three positions around the maximum STC index value.

FIGS. 17a and 17b are separation plots for 40 lesions showing the effects of amplitude normalization on separation of STC spectra of fibroadenoma and cancer lesions. Separation of 18 fibroadenoma and 22 cancer using weightings with p=10 wavelength points of the STC spectra. D_{c} and D_{f} refer to the “distance” or the “discrimination” from the average STC spectra for cancer and fibroadenoma, respectively. The “score” is the score calculated from algorithm for best weighting using equations as described below. The plot shows fibroadenoma, cancer, and reference points. FIG. 17a shows the plot with no normalization of amplitudes and FIG. 17b shows the plot with normalization of amplitudes.

FIG. 18 is a graph of the average STC spectra (double differential residual) for 22 malignant and 18 benign lesions at location exhibiting the largest variation in comparison to surrounding normal tissue on tumor-containing breast. Normal tissue obtained from averaging normal tissue at the equivalent position on normal contra lateral breast of the lesion patients and from the STC absorption spectra obtained for normal (i.e no known lesion) subjects.

FIG. 19 is a graph showing the average STC spectra for cancer, fibroadenoma and normal subjects. STC spectra averaged for 22 cancer lesion and 18 fibroadenoma lesions. These spectra have been amplitude normalized.

FIGS. 20, 20a-20c is a three part diagram showing STC spectra which are spatially localized, and lesion type specific. The left breast contains tumor. STC spectra for cancer and normal tissue are different. The box in the image of the breasts indicates the region of interest (the tissue region measured). Dots indicate points at which measurements were obtained. The STC spectra shown at two positions over tumor containing tissue (FIGS. 20a and 20b), and over normal tissue (FIG. 20c).

FIGS. 21, 21a-21c is a three part diagram similar to FIGS. 20, 20a-20c showing STC spectra which are spatially localized, and lesion type specific. The right breast contains lesion. STC spectra for fibroadenoma and normal tissue are different. The box in the images of the breast indicates the region of interest (the tissue region measured). Dots indicate points at which measurements were obtained. STC spectra shown at one position over tumor containing tissue (FIG. 21a), and over normal tissue (FIG. 21b).

FIG. 22 is a separation plot of benign and malignant lesions using spectral separation method which separates by maximizing differences in wavelength regions. Benign and malignant lesions separate with 100% sensitivity and 92% specificity. D_{c} and D_{f} refer to the “difference” or the “discrimination” from the average STC spectra for cancer and fibroadenoma, respectively. Points for fibroadenoma, and cancers are shown.

FIGS. 23a-23d are spectral absorption graphs which illustrate the steps of the double differential method.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

The illustrated embodiment is a double differential method to analyze the near-infrared spectra of regions of the human breast with tumors. We show that the near-infrared (650-1000 nm) spectra of breasts with tumors have characteristic absorption bands in the lipid fingerprint region that are unaccounted for in conventional spectral models. These spectral components do not appear in the normal breast of the same patient or in regions of the diseased breast away of the tumor. These spectral components originate from lipids that are present in tumors either in different abundance than in the normal breast or new lipid components that are caused by the different metabolism in tumors. Furthermore, the water band in the 980-1000 nm region also shows distinct variations in the tumor region compared to the normal breast. By combining the information in the lipid and water region, we constructed an index that is characteristic of the tumor region (100% specificity and 93% sensitivity for the 17 patients investigated) and has the potential to distinguish benign form malignant tumors on the basis of the lipid composition and/or bound water. This index can be combined with previously described indexes based in the amount of water, lipids and hemoglobin to further improve the diagnostic power of optical spectral methods. Surprisingly, the size of the tumor (range 4-70 mm) does not correlate with the value of this index, providing similar sensitivity for small and larger tumors.

In this disclosure we discuss a spectral continuum method to explore if there are specific spectral signatures that can differentiate normal tissue from tumors. We believe that these spectral signatures can only or best be revealed using a continuum or a substantially continuous range of wavelengths. It is well established that a series of cellular modifications occur during tumor growth, including regulation of protein synthesis, lipid synthesis and oxidation, angiogenesis and changes in the tissue water content. Whether or not some of these metabolic changes carry specific spectral signatures in the near-IR has been a matter of debate. Furthermore, it is unclear if we have the sensitivity to detect these potential signatures using non-invasive optical spectroscopy. The concentrations of some specific tumor metabolites could be below the sensitivity threshold of present spectroscopic techniques. It is generally agreed that extrinsic markers, either absorbing or fluorescent could provide a specific tumor signal, although it remains unclear how to deliver the specific markers to the tumor.

An extensive search for intrinsic spectroscopic markers has not been carried out to date. In our opinion a major conceptual difficulty arises because of the approach used to search for these spectroscopic signatures. Typically, the analysis proceeds by fitting the spectroscopic data to a series of pre-assigned basis spectra or by performing principal component analysis (PCA). The difficulty with the basis spectra approach is that the difference in breast composition, specifically lipid composition, produces mismatches between the experimental data and the fit, giving residuals that mainly reflect intersubject differences rather than tumor-normal tissue differences. In the PCA analysis, the finding that the lipid spectra change from patient to patient is of importance, but does not solve the problem of finding specific tumor signatures.

Here we propose a different approach in which only the spectral differences between normal and diseased tissue are analyzed, not the mismatch between actual optical spectra of the patient and the spectra of the data set used for fitting of the tissue components. This differential approach can be performed either by comparison between the normal breast and the breast with tumor in the case of unilateral tumors or between different parts of the same breast, presumably comparing regions with the tumor and regions without the tumor. The question which is addressed is: are there unique spectral differences between the normal and tumor-containing breast tissues besides spectral differences resulting from tissue composition, namely water, lipids (which are patient specific) and the two forms of hemoglobin? Although this question in principle can be answered by fitting the spectral absorption data with the basis spectrum of the tissue components, the mismatch of the actual spectrum of the tissue component and the assumed spectra of the data base may be larger than the subtle residual spectral differences which are characteristic of tumors.

Therefore we will proceed by first subtracting the spectrum of the normal breast from the spectra obtained at different locations in the breast with the tumor and then fitting the differential spectra using the basis component spectra and proceeding with the analysis of the residues of this fit. After this double differential operation (analysis of the residuals of the differential spectrum), we are able to detect subtle spectral features between the tumor region and the normal tissue.

Two crucial internal controls are performed for each patient. For the normal breast tissue, the fit of the difference between the average spectrum and the spectra at different locations (in the normal breast) should be accounted for by the “natural” compositional difference at different locations of the breast, but no new component should be needed. For tumor breast tissues, there should be location-specific residuals that cannot be accounted for by the four major tissue components. We note that the standard basis composition heterogeneity is still of great value in assessing the presence of the tumor. However, the analysis of the tumor-specific residuals will provide more specific information about anomalous biological processes and it will enhance contrast.

The Double-Differential Method of Data Analysis

Spectra at different locations in the breast are collected in the 650-1000 nm region using a conventional handheld frequency-domain scanner. This instrument collects spectral data in the wavelength range from 650 nm to 990 nm and additionally, frequency-domain data at six different wavelengths in the spectral range 670 nm to 860 nm. These frequency domain data, in conjunction with the equations for light propagation in the diffusion regime, are used to determine the spectral dependence of the tissue reduced scattering. Subsequently, the reflectance spectra are reduced to absorption spectra using conventional computational procedures. After data reduction from reflectance to absorption, all absorption spectra collected for the normal breast are averaged and then subtracted from the data at each location for the breast with the tumor as well as for the normal breast. In mathematical terms, the operations performed by our algorithm are detailed below.

Assume that the absorption spectrum at each location can be expressed by the linear combination of the basis spectral components with the addition of an unknown term specific of the tumor

$$S ( λ , x , y ) = {\sum }_{i} {a}_{i} ( x , y ) \times {S}_{i}^{*} ( λ ) + S T C ( λ , x , y ) \qquad Eq 1$$

where aᵢ are fractional contribution to the overall absorption, Sᵢ* are the basis spectra, which in the illustrated embodiment are water, lipid oxy and deoxy-hemoglobin are functions of wavelength λ, that can be patient specific and STC represents the residual tumor-specific spectral component that is not contained in the basis set and which is a function of wavelength λ and position x, y. We assume that STC is a small contribution. It is this component that we want to retrieve. The * in the experimental basis spectra indicate that these spectra could be patient-dependent.

The average experimental absorption spectrum A(λ) in equation #2 below in the normal breast is obtained by averaging the absorption spectra at different locations in the normal breast.

$$A ( λ ) = {\sum }_{i , x , y} {c}_{i} ( x , y ) \times {S}_{i}^{*} ( λ ) \qquad Eq 2$$

The differential spectrum at each location is obtained by subtracting the average absorption spectrum of the normal breast from the absorption spectra at each location, both for the tumor-containing and normal breast tissues:

$$\begin{matrix}D ( λ , x , y ) = {\sum }_{i} {a}_{i} ( x , y ) \times {S}_{i}^{*} ( λ ) + S T C ( λ , x , y ) - {\sum }_{i} {c}_{i} ( x , y ) \times {S}_{i}^{*} ( λ ) \\ or & Eq 3 \\ D ( λ , x , y ) = {\sum }_{i} {Δ}_{i} ( x , y ) \times {S}_{i}^{*} ( λ ) + S T C ( λ , x , y ) & Eq 4\end{matrix}$$

where the symbol Δᵢ indicates small differences between the spectra at different locations. We define the residual spectra

$$S T C ( λ , x , y ) = D ( λ , x , y ) - {\sum }_{i} {Δ}_{i} ( x , y ) \times {S}_{i}^{*} ( λ ) \qquad Eq 5$$

In the absence of the STC component, the residual spectra should be relatively small (ideally zero). The coefficients Δᵢ should indicate the different amount of the basis components in different regions of the breast. In order to estimate STC, we fit the residual function defined in equation 5 using a standard basis set for the major tissue components instead of S* which is unknown. If the STC component is absent, the fit should only show the differential concentration of the basis components in the different regions of the breast. Since the major mismatch between the patient specific average composition and the standard basis set has been accounted for by subtracting the average spectrum of the normal breast, we expect that the residuals should be small, even if we substitute the S* patient specific set with the standard set S. Of course, this statement can be verified experimentally using the data from the “normal breast”.

Note that by fitting the differential spectra rather than the absorption, the differences between the actual basis spectra for the patient and the spectra of the standard basis set is now minimized and the STC component can be recovered with relatively high precision. If we had fitted Eq 1 rather than Eq 5 using the “standard data set” the coefficients aᵢ of the fits would have been large and the difference between the specific spectra of the basis components in the patient with respect to the standard basis set will overwhelm the subtle differences due to the STC component. A second control/prediction is that in the breast with the tumor, the STC component should be present only in the tumor region. Also this prediction can be verified experimentally.

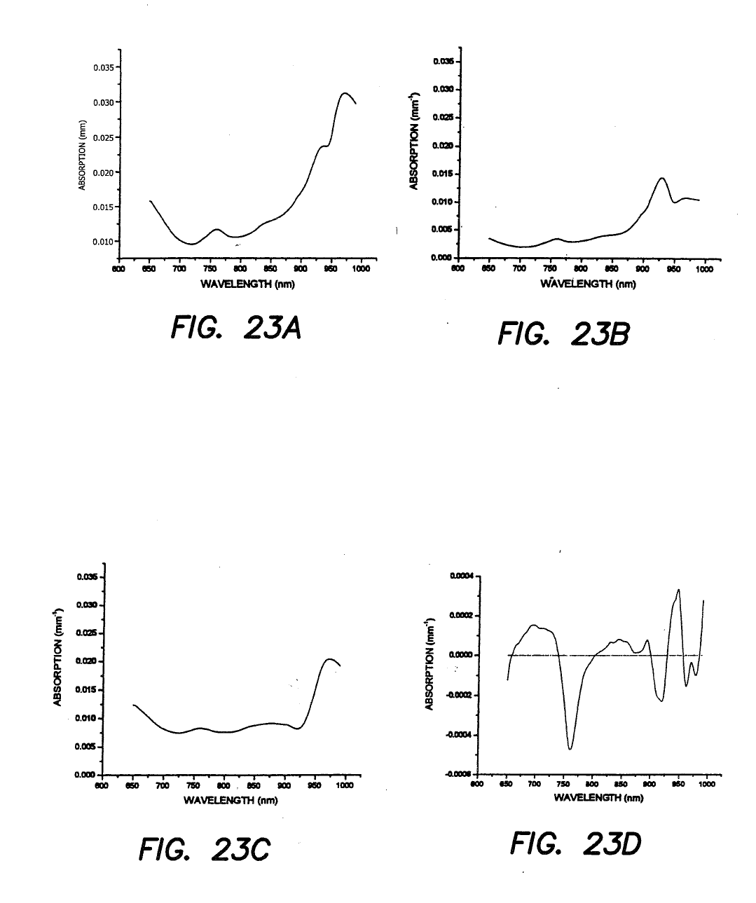

FIGS. 23a-23d illustrate the algorithm used in the double differential method described above as applied to absorption spectrum over tumor-containing tissue at one spatial location. FIG. 23a is a graph which shows the measured scatter-corrected absorption spectrum at a single spatial location over tumor-containing breast tissue. FIG. 23b is a graph which shows a representation of average measured absorption spectra over normal tissue. FIG. 23c is the difference spectrum obtained by subtracting the average absorption spectrum of the normal breast tissue in FIG. 23b from the absorption spectrum at a single spatial location over tumor-containing breast tissue in FIG. 23a. FIG. 23d is the STC spectrum obtained from subtracting the fit of the difference spectrum using the four basis components (oxyhemoglobin, deoxyhemoglobin, water and bulk lipid) used in the illustrated embodiment from the difference spectrum of FIG. 23c.

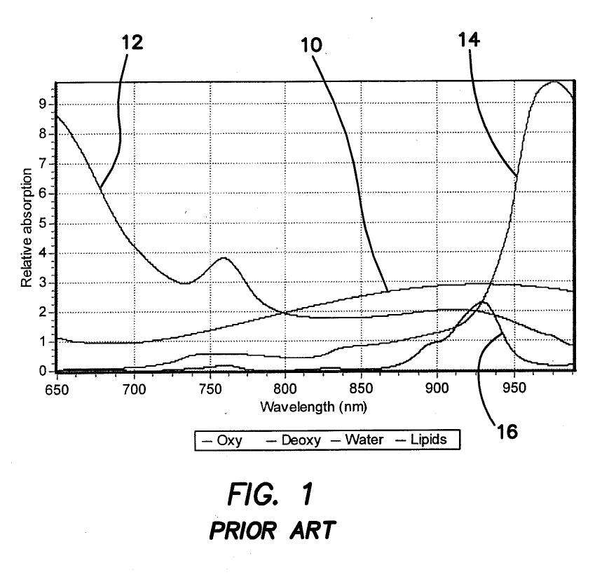

To validate our approach and to test that there are tumor-specific and spatially-localized spectral components in the breast, we need some sort of spatial resolution of the spectroscopic signal. A number of studies and theoretical predictions about light propagation in tissues have shown that by using a “reflectance geometry” in which light is injected at one point at the tissue surface and then collected using an optical fiber bundle at a distance of about 3-4 cm can produce optical signals with a spatial (voxel) resolution of about a cm. In principle, reconstruction of the light path could increase the contrast ratio of the method. For the purpose of this disclosure, we will simply assign the optical signal to the region in between the source and the detector fiber. The standard spectra set showing relative absorption as a function of wavelength between 650 nm and 1000 nm, i.e. oxyhemoglobin by graph 10, deoxyhemoglobin by graph 12, water by graph 14 and lipids by graph 16, used for the fitting of Eq 5 is shown in the FIG. 1.

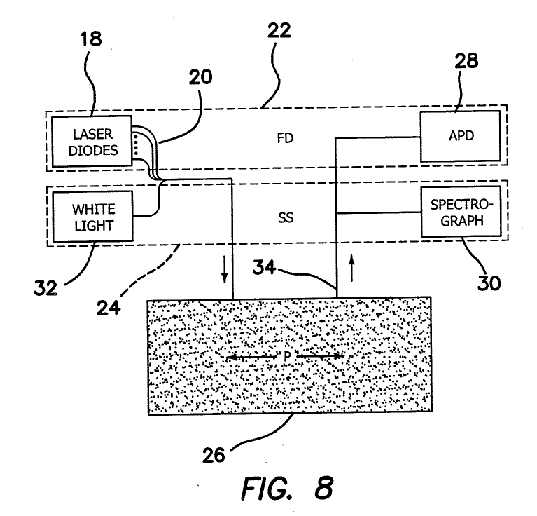

Consider now an experimental verification and proof of concept of the method disclosed above. Measurements were taken using the a conventional laser breast scanner (LBS) which combines frequency domain photon migration (FDPM) with steady state (SS) spectroscopy thereby increasing the spectral bandwidth to provide absolute absorption spectra in wavelength regions of 650-1000 nm. This technique is known as Steady State Frequency Domain Photon Migration (SSFDPM). Details describing the principles underlying the instrumentation method and theory have been described in the incorporated patent application above. The FDPM component uses the multi-frequency, single source-detector separation approach. In this embodiment of the LBS instrument as diagrammatically shown in FIG. 8, the FDPM component 22 employs six laser diodes 18 at the wavelengths of 658, 682, 785, 810, 830, and 850 nm. Other frequencies could be utilized consistent with the teachings of this invention. These laser diodes 18 are organized into a 3 mm diameter fiber bundle 20 of 400 μm fibers. Each wavelength delivers 10-20 mW of optical power to the tissue. During a measurement, the laser diodes 18 turn on serially to deliver light into the tissue 26. A network analyzer (not shown) is used to modulate the intensity of this light between 50 to 600 MHz in 2 MHz steps. After propagation though the tissue 26, the intensity-modulated light is detected by an avalanche photodiode detector (APD) 28. The network analyzer then compares the detected signal with a reference source signal. The final output corresponds to the phase and amplitude of the detected signal relative to the excitation light as a function of modulation frequency. While it takes a fraction of a second (200 ms) to sweep and acquire data over all of the modulation frequencies for a single wavelength, the total time to go through six laser diodes 18, transfer data, display data, and switch sources, the total acquisition time is approximately 20 seconds. The FDPM system 22 is calibrated using phantoms of known tissue properties at the beginning, middle and end of the measurement.

The steady state SS component 24 delivers broadband light from a high intensity tungsten-halogen lamp 32 by Mikropak. Changes in reflectance are measured using a 1 mm fiber 34 coupled to a spectrometer 30 providing 1-2 nm resolution. For the measurements reported in this disclosure, the BWTec and Oriel spectrometer provided sensitivity to 996 nm and 1000 nm, respectively. The SS system 24 was calibrated using an integrating sphere.

For both the FDPM 22 and SS 24, the source and detector in each system have been coupled into a black plastic covered handheld probe (not shown). The source and detector are positioned to be about 28-29 mm apart in linear distance. Furthermore, the beam paths between the source and detector in each component 22, 24 have been placed in an “X” configuration such that the measurement paths cross, thus allowing both methods to interrogate nearly the same tissue volume. Generally a SSFDPM measurement takes on the order of 30-45 seconds, and a complete session of measurements takes around 45 minutes depending on the number of measurements, difficulty in locating the lesion, technical difficulties, etc.

Data Processing

Raw data were analyzed using custom-made software with a MATLAB platform. For each measurement position on the breast, the data corresponding to the phase and amplitude data from the FDPM 22 and the reflectance spectra from the SS 24 were processed according to the algorithms described in the incorporated application above to recover the complete scattering and absorption spectra for the full bandwidth of 650-1000 nm.

Processing begins by fitting the frequency-domain data to a P1 approximation to the radiative transport equation to determine the μₛ′ and μₐ of the tissue. In the fitting procedure data are fit from 50-550 MHz, with specific source detector distances as noted above during data acquisition for the FDPM 22 and SS 24 set-up. A Levenberg-Marquardt minimization algorithm is utilized to minimize the chi-squared function for the real and imaginary portions of the output signal, which are magnitude-balanced transforms of the measured phase and amplitude. The final results are absolute μₛ′ and μₐ values at each of the laser diode wavelengths.

Two procedures are then carried out to determine the absolute scattering and absolute absorption spectra. For scattering, the wavelength dependence of the scattering is assumed to have particular form of μₛ′=Aλ^{-SP}, where A is the amplitude and SP is the scatter power. Data at the six wavelengths of the laser diodes 18 are constrained to follow this relationship from which the constants A and SP are determined. This procedure provides the scattering spectrum. To recover the absorption spectra, a theoretical reflectance is calculated using μₛ′ and μₐ obtained at the 6 different wavelengths from the frequency-domain data. The experimental reflectance data is then normalized by a constant to this calculated reflectance spectra. Using the scattering spectra, the normalized experimental reflectance is calculated in the whole spectral range from 650-1000 nm. For the analysis described in this disclosure, it is this absolute absorption spectrum obtained by the above procedure which is utilized in the double differential spectroscopic method.

The absorbance data were analyzed by a conventional Elantest program using equations 1-5 above to calculate the STC component. The fit of the differential spectra was done using a linear combination of the standard data set shown in FIG. 1.

Measurement Procedure

The patients were asked to fill out a patient history form including information such as age, menopausal status, family history, etc. After gaining written permission, ultra sound and surgical pathology reports are utilized to determine the type, localization, and extent of the tumor lesion. We then combined these data with palpation for final determination of the measurement locations.

For the measurement session, the patient was asked to lie in a comfortable supine position. Using a surgical pen measurement positions were marked at 1 cm intervals in a line across the tumor location including some normal surrounding tissue. The line scan markings do vary across the patients; some subjects were marked in an “X” configuration, while others were measured with either a single line or three parallel lines. Nevertheless, in all cases the tumor region was measured.

Using the handheld probe, measurements were taken in reflection geometry, with particular care to maintain the probe in contact with the surface skin without compression. For control purposes, measurements were also taken in the same locations on the contralateral breast.

Results

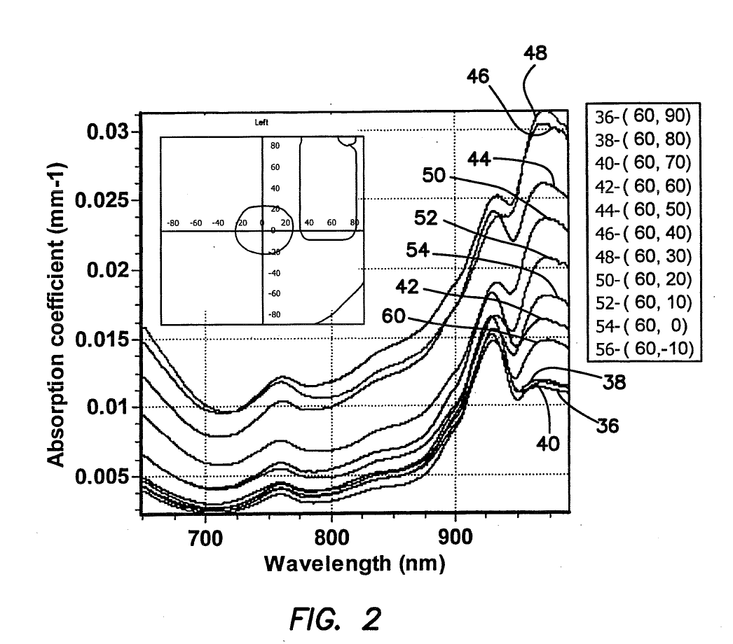

FIG. 2 shows the absorption spectra obtained at discrete spatial locations along a line in the breast with tumor for patient #30 (see Table 1 below) at the positions along the vertical line shown in the inset in the upper left portion of the graph of FIG. 2, where the absorption coefficient is graphed as a function of wavelength. The darkened region in the inset represents the amount of the water component in the breast with the tumor, relative to the average water content of the normal breast of the same patient. The coordinates in the inset are in millimeters. The legend on the right of the figure shows the positional coordinates 36-56 where the spectra were measured. The amount of the water component was obtained by fitting the absorption spectra with the basis set shown in FIG. 1. It was assumed that the experimental spectra can be described by the linear combination of the basis spectra.

TABLE 1

| | | Tumor | | | |
|---|---|---|---|---|---|
| Age | Menopausal | classifi- | Size | | |
| (years) | Status | cation | (mm) | STC Tumor | STC Normal |
| 47 | PRE | DC | 24 | 120.1 | 15.0 |
| 38 | PRE | DC | 24 | 100.2 | 9.7 |
| 50 | POST | AC with | 54 | 216.9 | 4.4 |
| | | L.F | | | |
| 32 | PRE | DC | 29 | 422.9 | 51.5 |
| 57 | POST | DC w/ | 32 | 115.5 | 20.0 |
| | | L.N.M | | | |
| 47 | POST | DC | 17 | 609.3 | 8.4 |
| 45 | PRE | DC | 29 | 162.5 | 7.8 |
| 44 | PRE | DC | 16 | 62.7 | 12.9 |
| 49 | POST | DC | 70 | 148.3 | 9.3 |
| 41 | PRE | DC | 40 | 86.1 | 4.2 |
| 53 | POST | DC | 24 | 132.3 | 6.3 |
| 57 | POST | DC | 31 | 135.2 | 4.1 |
| 47 | PRE | DC | 24 | 120.1 | 15.0 |
| 38 | PRE | DC | 24 | 100.2 | 9.7 |
| 50 | POST | AC with | 54 | 216.9 | 4.4 |
| | | L.F | | | |
| 32 | PRE | DC | 29 | 422.9 | 51.5 |

The darkness of the portions in the figure inset corresponds to the differential amount of water in the tumor region with respect to the average of the normal breast.

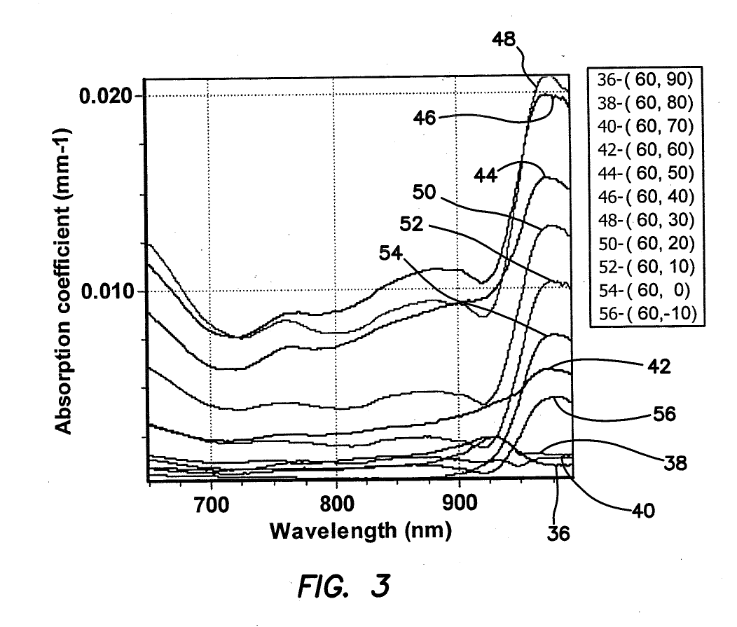

FIG. 3 shows the differential spectra corresponding to equation 3 for patient #30 using the average spectra of the normal breast as the reference spectrum. The differential spectra show that along the lines of measurement, the tissue is not homogeneous. However, without further analysis, it is difficult to say if the differential spectrum solely reflects different amounts of the four basic components or has some extra features. In fact, the differential spectra at different locations for the normal breast show similar broad features.

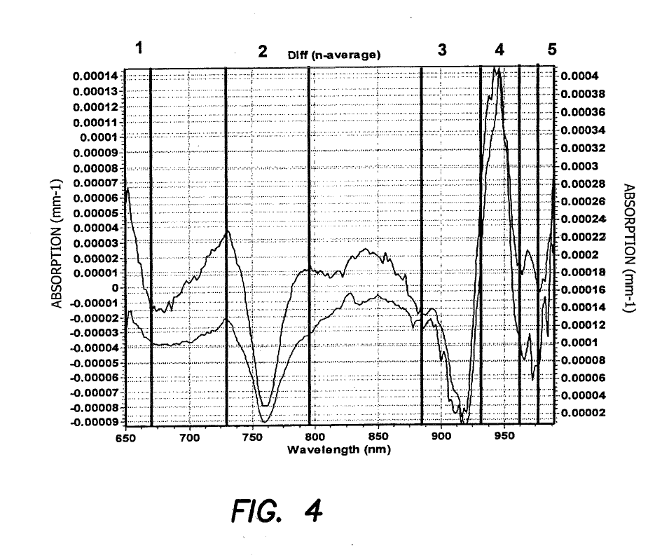

FIG. 4 is a graph of absorption as a function of wavelength which shows the residuals after the fit using the four tissue components basis spectra shown in FIG. 1. The tissue components used for the fit were water, lipid, oxy and deoxy-hemoglobin, as reported in FIG. 1. Five regions where changes are more noticeable are identified in the figure with numerals 1-5 across the top of the graph. The residual spectra correspond to the 11 positions along the line shown in the inset of FIG. 2.

The residuals show definitive patterns, although their amplitude is relatively small (about 1% of the original spectra). When compared to the residual obtained when the same series of operations are applied to the normal breast, the residuals for the breast with the tumor are at least one order of magnitude larger (Spectra not shown, numerical data in Table 1). The residual spectra shows characteristic peaks while the residuals for the normal breast are randomly distributed.

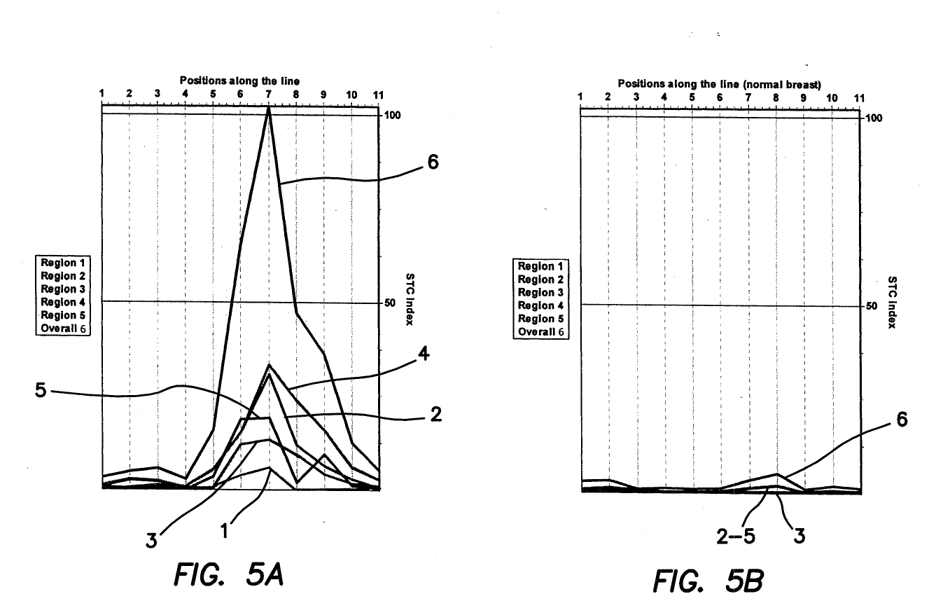

Inspection of the STC component shown in FIG. 4 reveals that there are roughly five regions (identified in FIG. 4 with numerals) where systematic differences are observed. To quantify the magnitude of the residuals in these five regions we calculate the local residual variance defined by

$${L}_{k} ( x , y ) = \frac{{\sum }_{i , k} {( S T {C}_{i} ( λ , x , y ) - \overline{S T C} ( x , y ) )}^{2}}{{N}_{k}} \qquad Eq 6$$

where the index k indicates wavelength values in the five spectral regions and Nₖ indicates the total points in each of the five regions. FIGS. 5a and 5b show the values of the local residual variance at 11 positions in the breast with tumors along the line indicated in the inset of FIG. 2, for the diseased and for the normal breast (Patient #30), respectively. The sum of all L values at the five spectral regions is also shown in FIG. 5a for the breast with tumor and in FIG. 5b for the normal breast. The values of the local residual variance at the 11 positions is dramatically different.

A total of 17 patients presenting different tumors were analyzed using the differential spectroscopic method described above. In all cases the STC component in the breast with tumor was substantially larger than the STC component in the normal breast. Table 1 above reports the maximum value of the sum of the STC components for the patients investigated for the normal and for the breast with tumor as well as the classification and size (from ultrasound) of the tumor as obtained from pathology.

Comparison Between TOI and STC.

It was previously proposed that tumors can be optically detected using a combination of tissue components known as the Tumor Optical Index (TOI) value. This TOI value corresponds to the following product

$$T O I = \frac{Water \times Deoxyhemoglobin}{Lipid} \qquad Eq 6$$

For the 17 patients investigated we also calculated the maximum TOI value in the tumor side and in the normal breast (See Table 1). Although there is excellent correlation between the STC and the TOI values, the STC value has better specificity and sensitivity. For the 17 patients in Table 1, using the contralateral breast as the negative control, we estimated that the TOI parameter has a sensitivity of 73% and specificity of 100%. For the STC value the sensitivity is 93% and specificity is 100%. The major difference between the TOI and the STC is that the TOI is based on the abundance of tissue components (water, deoxyhemoglobin and lipids) at different locations in the breast, while STC is based on the presence of specific spectral components. The two indexes can be combined to obtain a better discrimination of the regions of the breast with tumors.

Possible Biochemical/Physical Origin of the STC.

For the STC parameter to be interpreted in physiological terms, we propose the following possible origin of the STC component. Region 1 corresponds to the hemoglobin or melanin absorption region. At present we cannot uniquely identify the spectral origin of the STC bands in region 1. We propose that the feature in region 2 (FIG. 4) is due to disappearance of a specific lipid component at the tumor location or due to broadening of the lipid band at this location. Note that according to equation 5, a negative peak of the residual indicates the lack of an additional spectral component. Possible candidates are a change in the cholesterol content or increase in lipid oxidation with broadening of the band that has been proposed to be more abundant in tumors. The features in region 3 and 4 (FIG. 4) can be a combination of spectral shifts and changes in lipid composition in the tumor. This is the spectral region in which lipids have the largest absorption in the near-IR and it is likely that if there are differences in lipid composition in the tumor with respect to the normal tissue, this spectral region will be affected. All tumors have a characteristic behavior in this region, starting with a negative peak in region 3 followed by a large positive peak in region 4. In some patients, there is also a negative band at longer wavelengths. This characteristic oscillation of the residual could be due to a narrowing of the major near-IR absorption band of the lipids or due to a combination of spectral shifts. Region 5 (FIG. 4) is probably due to changes in the water spectrum. Two effects are known to change the spectrum in this region, namely i) the change in the relative amount of bound water and ii) temperature changes associated with the increased metabolism in the tumor. Both changes have been previously proposed as characteristic of tumors. As the temperature is increased, the near-IR band of water moves toward shorter wavelengths giving rise to a characteristic shape for the differential spectrum with a positive peak at 980 nm followed by a negative peak in a region which is outside the wavelength range measured in this study. Instead, an increase of the amount of bound water gives an opposite behavior. The experimental results for the STC component show a negative shoulder in the 980 region followed by a positive peak at 990 nm. This behavior is compatible with a decrease of the amount of bound water component in the tumor as compared to the normal tissue.

Correlation Analysis

To better identify the origin of the STC component and to further discriminate between the appearances of specific lipid components in different type of tumors, we calculated the correlation between the changes in the spectral regions 1 to 5. For the patients analyzed, region 4 always correlates with the changes in region 3, although with ratios that are patient (and presumably tumor) dependent. Instead, the changes in region 1 and region 5 have less evident correlation with the other regions. Of course, the number of patients analyzed is probably too small to confidently statistically classify different types of tumors based on these correlations.

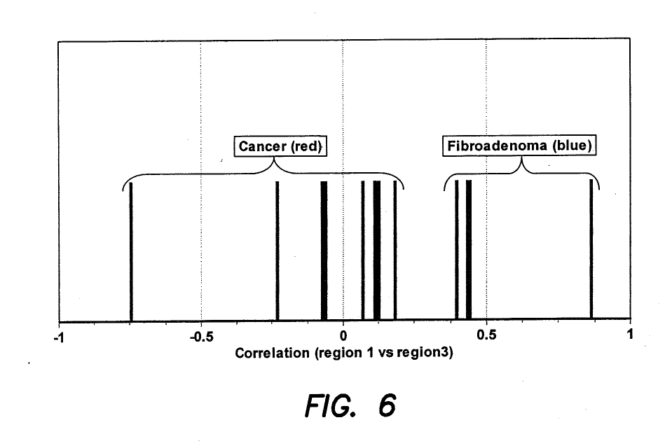

We show in the graph of FIG. 6 the values of the correlation ratio between region 1 and region 3 for the 12 patients with cancer (reported in Table 1) and 4 patients with fibro adenoma, a benign tumor. Clearly, the fibro adenoma show a larger positive contribution of the STC component in region 1 that the cancer cases. This example shows that the different parts of the STC component carry independent information about the type of tumor,

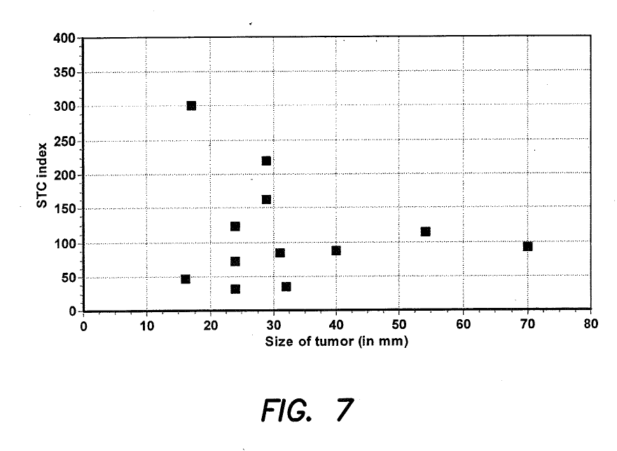

We also evaluated the correlation between the maximum value of the STC component and the size of the tumor (See Table 1). FIG. 7 shows the correlation plot. Surprisingly, there is no apparent correlation between the maximum of the STC component and the size of the tumor. We believe that some correlation should exist, at least for the very small tumors. What we have not done is to correlate with the depth location of the tumor, since this information was not available in the reports in this study.

In summary, by using a double differential spectral method we have demonstrated the existence of specific spectroscopic signatures that are characteristics of tumors. These signatures are spatially localized to the regions of tumor-containing breast. We believe that these characteristic tumor signatures arise because of different lipid type and bound water composition in the tumor region. The observation of specific tumor spectral signature opens new possibilities for the application of optical methods for the early detection of breast tumors and brings optical biopsy closer to its full realization. The interpretation of the spectral signature with changes in the lipid composition leads to the use of the STC component as a related indicator of tumor oncology in which the changes in lipid regulation are direct evidence of breast diseases.

Spectral Separation Method

Consider now a feature analysis method which exploits the entire STC spectra to achieve discrimination of benign and malignant lesions. The equations for the spectral separation method and optimization algorithm used discrimination are given below. The STC index (a number) provides an easy and quantitative way to identify the presence of a lesion. However, the STC index is insufficient to separate benign and malignant lesions. Preliminary analysis on fibroadenoma STC spectra revealed variations in the same regions as noted for cancers (650-665 nm, 730-800 nm, 875-930 nm, 930-960 nm, and 980-1000 nm), and of comparable amplitude resulting in a similar index value. Thus we needed a different method that exploits the entire STC spectra to achieve separation.

The idea is to separate lesion types by maximizing the differences of sub-indexes calculated in different spectral regions. We began by looking for different combinations of indexes, including sums, ratios, and multiplications. In examining a set of 24 lesions, 12 cancer and 12 fibroadenoma, we found that by using the ratio of the local variation of region 1 (650-665 nm)/region 3 (875-930 nm) R1/R3, we were able to obtain a sensitivity of 100% and specificity of 67% in the discrimination between fibroadenoma and cancer lesions.

Upon further examination, we found that this discrimination could be improved by using the value of the variation value for region 4 (930-960 nm) R4. The combination of the ratio of the two parameters, R1/R3 and R4, improved sensitivity and specificity to 100% and 92% respectively.

We then tried this analysis on a larger set of patients, but were unable to maintain such values for sensitivity and specificity. Our simple formulas were just describing the most visible differences we were noting in the STC spectra. This was surprising because it seemed that by simple inspection we could determine whether or not a given STC spectra could be classified as malignant or not. After examining many cases we had become trained readers. In effect we could recognize what the average STC spectra for cancer and fibroadenoma were, and thus could make judgments. This prompted us to ask the following question: how can we quantify the difference between a given STC spectrum from the average fibroadenoma STC spectrum and the average cancer STC spectrum in an analytical manner that is operator or physician independent?

Turn now and consider the spectral separation method in detail. We have developed a method to separate two types of spectra by calculation of the distance of a given spectra from the average spectrum of each type. This concept is based on the idea that a given cancer STC spectrum should be more similar to the average STC spectra for a set of cancer patients as opposed to the average fibroadenoma STC spectra. Thus the “distance” between a given cancer STC spectra and the average cancer STC spectra should be smaller than the “distance” between a given cancer STC spectra and the average fibroadenoma spectra. It follows that, most, if not all, cancer STC spectra and fibroadenoma STC spectra should group together and/or separate. In order to account for spectral differences across the full wavelength region of 650-1000 nm, the “distance” is calculated at each wavelength point at which data was obtained. Furthermore, in order to maximize the differences, wavelength regions are weighted. This method was then applied to separate STC spectra of benign and malignant lesions.

Consider the mathematics used to make the above quantification. Here we present the algorithm for separating two types of spectra. We assume that every patient can be represented by the STC absorption spectrum. First, we calculate the average STC spectrum of all known fibroadenoma cases, S_{F} and all cancer cases, S_{C}. Then we calculate the distance D_{F} (a number) to the average STC spectrum of a fibroadenoma and the distance D_{C} to the average STC spectrum of a cancer for a given patient.

$${D}_{F} = \frac{\sqrt{{\sum }_{i = 1}^{k} {( {S}_{i} - {S}_{F} )}^{2}}}{k}$$

$${D}_{C} = \frac{\sqrt{{\sum }_{i = 1}^{k} {( {S}_{i} - {S}_{C} )}^{2}}}{k}$$

For every patient, D_{F} is the distance of a given spectrum, Sᵢ, to the average STC spectrum of a fibroadenoma, S_{F}. Index i indicates a single wavelength point, and k represents the total number of wavelength points at which absorption data were obtained for the given spectrum. A similar calculation is made for D_{C}, the distance of a given spectrum, Sᵢ, to the average STC spectrum of a cancer spectrum, S_{C}.

Limit values for D_{F} and D_{C} are determined by substituting S_{C} and S_{C} into Sᵢ, and then calculating D_{Freference} and D_{Creference} values. For a given set of STC spectrum from patients, the value of D_{Freference} represents the average distance of a cancer STC spectrum to the average fibroadenoma STC spectrum. The value of D_{Creference} represents the average distance of a fibroadenoma STC spectrum to the average cancer STC spectrum.

From this calculation, the reference coordinates (D_{Freference}, 0) and (0, D_{Creference}) are plotted on a x-y coordinate system. In this graph the x axis represents the distance of a given spectrum from the average STC spectrum of a fibroadenoma, and the y axis represents the distance of a given spectrum from the average STC spectrum of cancer. The x and y axis are in μₐ absorption units of (mm⁻¹). For every patient, Sᵢ spectrum, D_{F} and D_{C} are calculated and plotted on the graph (as D_{F}, D_{C} coordinates).

Position on this map provides an indication of how similar and/or dissimilar a lesion is to the “average” fibroadenoma and cancer lesion. Spectra with x ordinate values closer to 0 and y ordinate values larger than D_{C} are more similar to the average STC spectrum of a fibroadenoma. On the other hand, spectra with x ordinate values greater than D_{F} and y ordinate values close to 0 are more similar to the average STC spectrum of a cancer. From hereon, the x ordinate will be referred to as the D_{F} term and the y ordinate as the D_{C} term.

Now consider the weighted D_{F} and D_{C} coordinates. In the D_{F}, D_{C} expressions all wavelength regions were treated equally. However, we noticed that only in restricted wavelength regions the differences between fibroadenoma and cancer were more evident. To maximize the separation of STC spectra from fibroadenoma and cancer D_{Fweighted} and D_{Cweighted} are determined by weighting the spectra in wavelength regions of Δλp, where p is the number of wavelength points in a group.

$${D}_{Fweighted} = \frac{\sqrt{{\sum }_{i = 1}^{k} {( [ {S}_{i} - {S}_{F} ] * {w}_{{F}_{Δλ p}} )}^{2}}}{k}$$

$${D}_{Cweighted} = \frac{\sqrt{{\sum }_{i = 1}^{k} {( [ {S}_{i} - {S}_{C} ] * {w}_{{C}_{Δλ p}} )}^{2}}}{k}$$

The value of p is variable and can be chosen by the user. Weighting factors for STC spectra of cancer, w_{CΔλ}p, and STC spectra of fibroadenoma, w_{FΔλ}p are determined using computer processing without operator or physician intervention through an iterative process for each wavelength region to determine what combination of values for each wavelength region would best separate the (D_{F}, D_{C}) coordinates of the fibroadenoma from the cancer lesions.

The “best weighting” is determined by minimization of a score, which is defined as follows:

$$score = {score}_{F} + {score}_{C}$$

$${score}_{F} = [ {( \frac{{D}_{F}}{{D}_{Freference}} )}^{4} + {( \frac{{D}_{Creference}}{{D}_{C}} )}^{4} ]$$

$${score}_{C} = [ {( \frac{{D}_{Freference}}{{D}_{F}} )}^{4} + {( \frac{{D}_{C}}{{D}_{Creference}} )}^{4} ]$$

score_{F} is calculated only for the fibroadenoma lesions and score_{C} is only calculated for only the cancer lesions.

The score is minimized by the computer algorithm by changing the weights. The result of this process is the “best weighting spectral vector” that maximally separates fibroadenoma from cancer.

Thus far the STC spectra used for separation are of different amplitudes. Here we normalize the amplitude to determine whether spectral shape can be used to discriminate benign and malignant lesions. The normalized D_{F} and D_{C} coordinates are determined by starting with a normalized STC spectrum. The normalization factor is n_{f} and n_{c} respectively for the fibroadenoma and cancer cases.

$${n}_{f} = \sqrt{\frac{{\sum }_{i = 1}^{k} {S}_{i}^{2}}{k}}$$

$$n c = \frac{\sqrt{{\sum }_{i = 1}^{k} {S}_{i}^{2}}}{k}$$

The weighted, normalized D_{F} and D_{C} coordinates are:

$${D}_{Fweighted , normalized} = \frac{\sqrt{{\sum }_{i = 1}^{k} {( [ \frac{{S}_{i}}{{n}_{f}} - {S}_{F} ] * {w}_{{F}_{Δ λ p}} )}^{2}}}{k}$$

$${D}_{Cweighted , normalized} = \frac{\sqrt{{\sum }_{i = 1}^{k} {( [ \frac{{S}_{i}}{{n}_{c}} - {S}_{C} ] * {w}_{{C}_{Δ λ p}} )}^{2}}}{k}$$

By using this option in the software program the amplitude differences between STC spectra of fibroadenoma and cancer are normalized thereby leaving the main source of difference to be the shape of the spectra.

Consider now the analysis of two sets of data, one of 24 lesions and the other of 40 lesions. This has been done for historical reasons as these two datasets were the original ones which were earlier reported. The main difference is the source of the data. In the data set of 24 lesions, data from University of California-Los Angeles (UCLA) Olive View Medical Center was included. With regards to the dataset of 40 lesions, we report on 40 lesions measured at the Beckman Laser Institute and Medical Clinic at the University of California-Irvine over dating from August 2004 to January 2007. For the purpose of publishing we excluded data from olive View. The set of 40 includes 17 patients from the first set of 24 lesions.

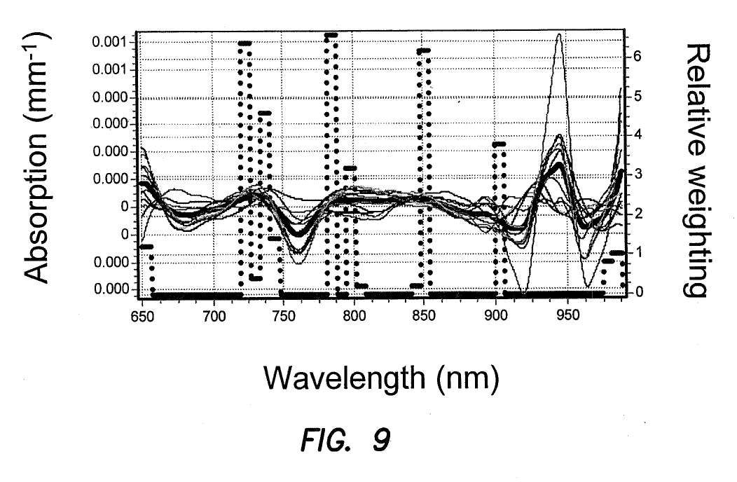

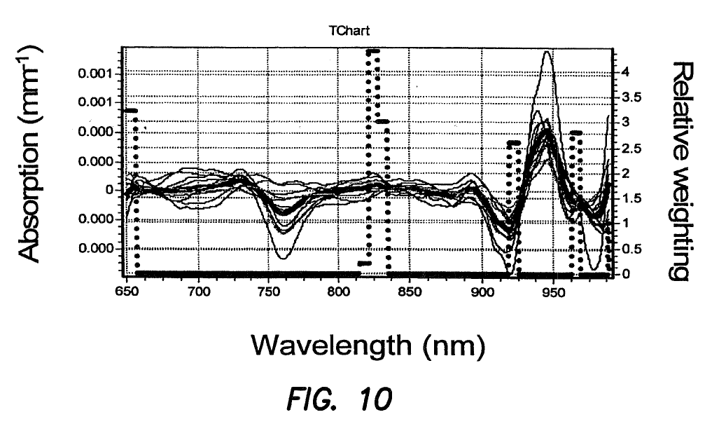

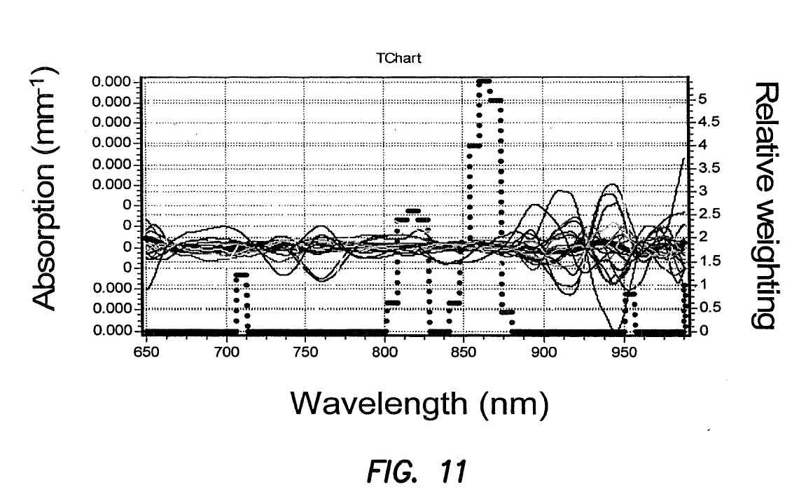

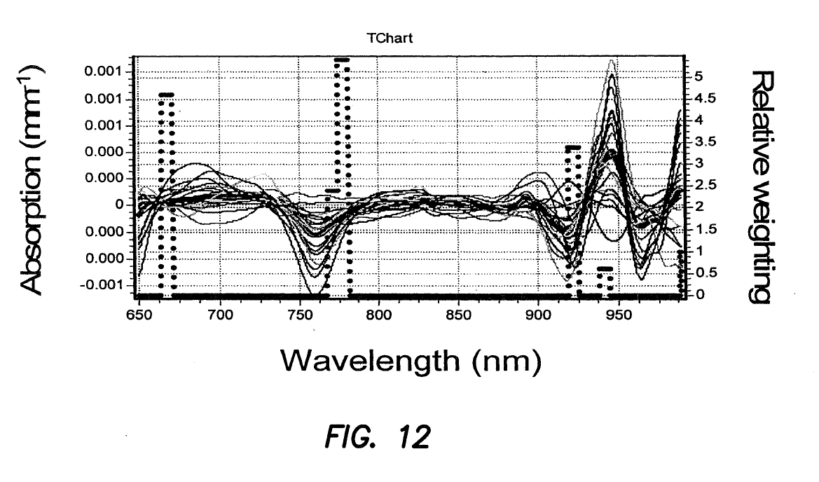

In FIG. 9 and FIG. 10 we show the STC spectra for the dataset of 24 lesions, 12 fibroadenoma and 12 cancers. In FIG. 11 and FIG. 12 we show the STC spectra for the dataset of 40 lesions, 18 fibroadenoma and 22 cancers. For both sets of data weightings were done in 20 wavelength point segments on STC spectra obtained at the position of highest variation. The left axis gives absorption (mm⁻¹), while the right axis indicates the relative weightings used to best separate the lesion types.

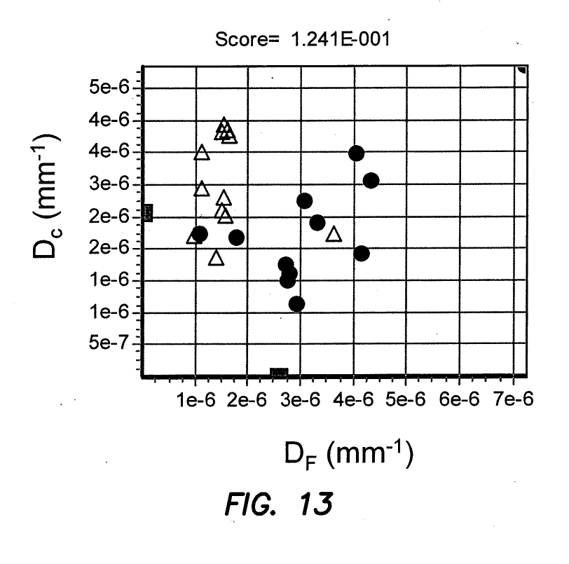

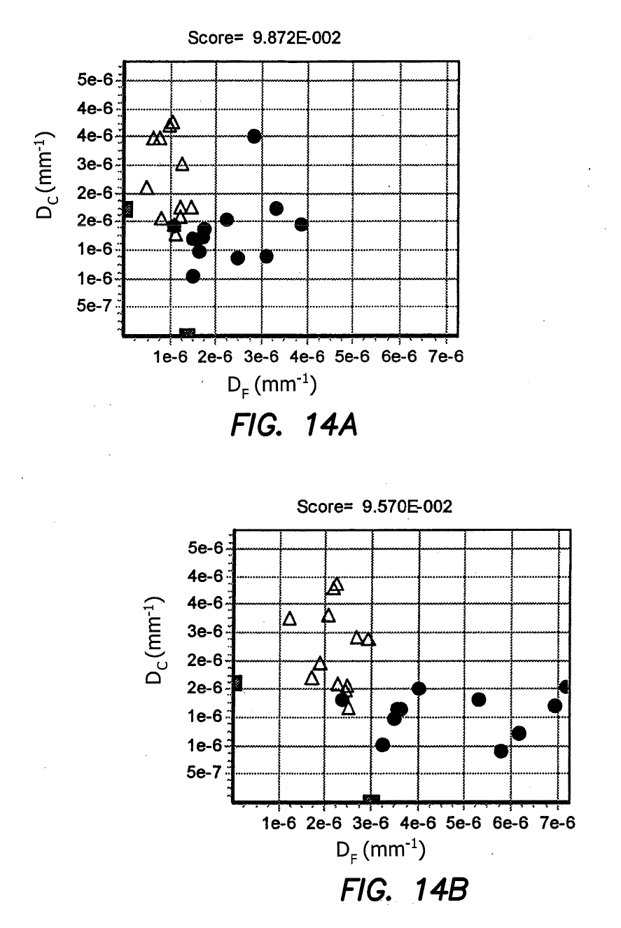

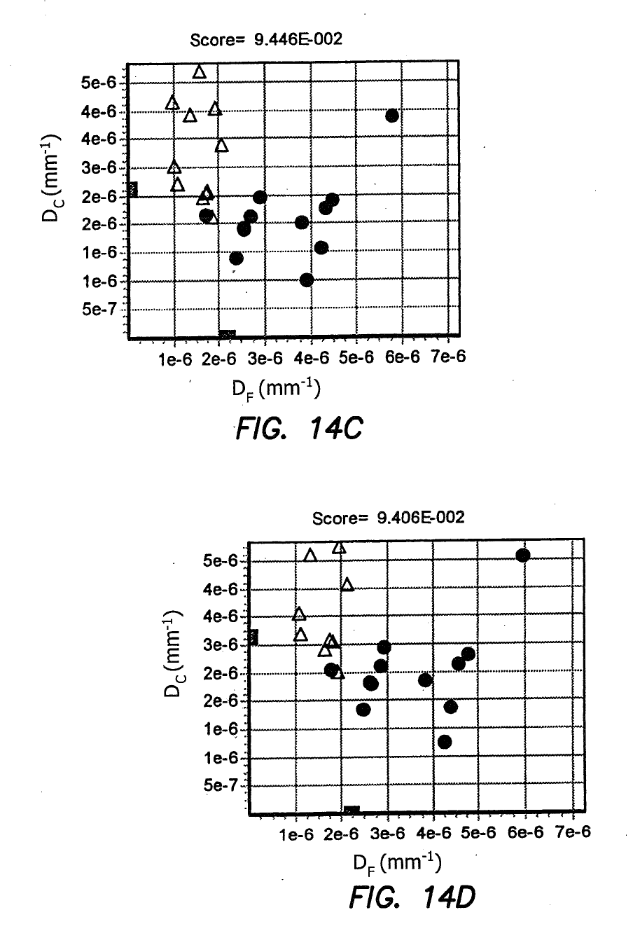

In FIG. 13 we show an example of the separation plot for 12 fibroadenoma and 12 cancer lesions using uniform weightings of the wavelength regions of the STC spectra. Note that for this analysis STC spectra were used from the positions exhibiting the highest STC variation (i.e. STC index value). In FIG. 14 we show the results for the same set of data presented in FIG. 13 after weighting optimization. (Note that 5 wavelength points represents about 2 nm). We systematically changed p values from 40 to 20, 10 and 5 points. We find there is better separation of the lesions as we increase the wavelength points. Note that for this set of spectra it is not necessary to set the p value to a lower value than 20 as we see good separation of data with p of about 20 (8 nm bandpass).

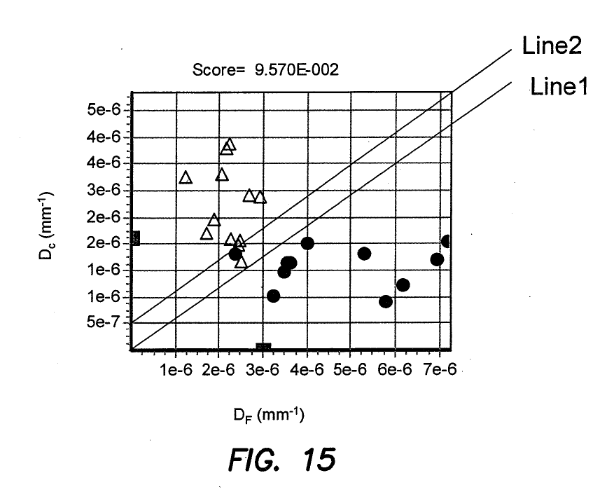

Here we choose to evaluate the data with p=20 as an example for evaluation of separation of fibroadenoma and cancer. We note that it is arbitrary to as to how the fibroadenoma and cancer patients are grouped, either by drawing a circle around each respective group, or perhaps a line of unity slope from the origin. For this data, if we draw a line of separation such as a line of unity slope (labeled Line 1), we obtain a sensitivity of 92% and specificity of 100%, whereas if we shift the line upwards (labeled as Line 2), then we obtain 100% sensitivity and 92% specificity. See FIG. 15. In this set of 24 lesions, we find clear separation of the fibroadenoma and cancer except for two lesions: one fibroadenoma and one cancer lesion overlap in space, in other words they are misclassified.

The optimization algorithm was applied to a larger data set of 40 lesions, 18 fibroadenoma and 22 cancer. For this dataset, we explored the best separation by exploring three parameters: the location at which the STC spectra were obtained, the number of wavelength points per region to determine the p points for weighting, and the normalization or lack of in the weighting procedure. Ultimately we found best separation when using a complete “line scan” of data, weighting was performed in 5 wavelength point segments, and the spectra were normalized. (As mentioned earlier, a complete “line scan” is a line of points at which data was obtained. This “line scan” extends from normal tissue on one side of the lesion, over the lesion, and to the other side.)

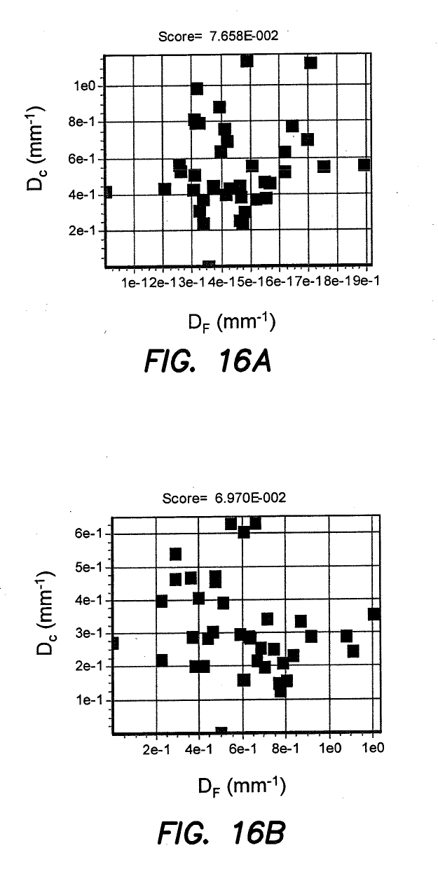

If we now compare results displayed in FIG. 16a in which the complete line scan of points were averaged to those shown in FIG. 16b in which the three points around the location of the maximum STC index were averaged, we find that the two lesion types separate with less overlap in FIG. 16a, while overall the two types of lesions separate farther from each other in FIG. 16b. We claim to find better separation in FIG. 16a, where we obtain 92% sensitivity and 100% specificity, whereas in FIG. 16b we obtain 87% sensitivity and 90% specificity using a line of unity as the divider between fibroadenoma and cancer lesions.

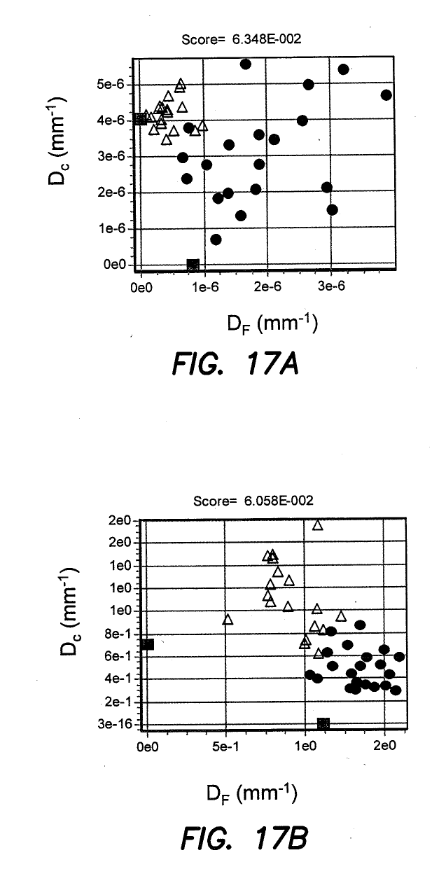

In FIGS. 17a and 17b we see the effects of amplitude normalization on separation using the spectra at the position of the highest STC variation. Note that for this example we have chosen to weight in spacing of 10 wavelength points. In FIG. 17a we see that before normalization, the points representing fibroadenoma seem to cluster near the reference point for the average fibroadenoma, whereas the points for the cancer seem to show more variation. If the spectra are normalized before weighting, shown in FIG. 17b then we see that the points for the cancer cluster close to the average cancer spectra, whereas the points for the fibroadenoma show more variation. Thus it seems that when using the spectral shape and amplitude together, the fibroadenomas are more similar to each other, and the cancers have more variation. But using only the spectral shape (and not the amplitude), the cancers seem more similar, and the fibroadenoma exhibit more variation.

In this study our aim was to differentiate benign and malignant lesions using only the STC absorption spectrum. Application of the spectral separation method on the STC marker has revealed that fibroadenoma and cancer lesions can be discriminated by using the STC spectra. Separation was obtained by spectral shape, the amplitude or amount of the STC absorption spectrum is not necessary. This is different from the traditional methods of analysis whereby tumors are separated by thresholding of tissue components present in both benign and malignant lesions using hemoglobin.

While we found that spectral shape is sufficient for separation of fibroadenoma and cancer lesions, both spectral shape and amplitude can be used. As shown is FIG. 17, using both spectral shape and amplitude the fibroadenomas cluster more towards the average fibroadenoma STC spectrum, whereas the cancer lesions show more variation. We believe that this is due to the amplitude difference in the spectra from the two lesion types. After normalization of the amplitudes, the cancer STC spectra cluster more closely to the average STC spectrum of cancer, whereas the STC of fibroadenoma lesions display more variation. See FIGS. 17a and 17b. With or without normalization, the two types of spectra separate.

Furthermore, these results suggest an interesting opportunity for monitoring and management of benign lesions. Given that the fibroadenoma lesions appear to be more variable across patients, in the future we can envision analyzing the STC spectra at a set of points of an area over the benign lesion tissue. After inputting these spectra into the spectral separation algorithm, the locations of these spectra can be tracked on the “map” of distances from average STC spectra of fibroadenoma and cancer lesion. Such “tracking” can facilitate physicians in monitoring of benign lesions, paying careful attention if and when a benign lesion moves to the “region” of malignancy on the map.

Preliminary exploration of the regions of weighting suggest which spectral regions are most important for separating the STC spectra of benign and malignant lesions. Although the algorithm iteratively processes through the full spectral bandwidth from 650-1000 nm, weightings are only needed at a few (5-10) regions. Following our operator-independent initial evaluation, region 1 (650-665 nm), region 2 (730-800 nm), region 3 (930-960 nm), and region 4 (960-980 nm) and region 5 (980-1000 nm) seem to be important. The weightings in regions surrounding major peaks, located near 760 nm, 920 nm, and 940 nm, seem to bring out the separation. This is interesting as these are regions where lipids are known to absorb. Differences near these peaks may be suggestive of differences in lipid types or metabolism. In addition, there appears to be a region from 820-870 nm and 970 nm which seems to be of importance. We note that the exact regions of weighting are different for the two sets of data, but the amounts are on the same order of magnitude. Nevertheless this can be used as an additional clue towards unraveling the biochemical origins of the STC spectrum.

At this stage of development we are in need of a larger dataset, on the order of hundreds, if not one thousand, to obtain the combination of weighting factors for best separation of fibroadenoma and cancer lesions using the STC absorption spectrum. Here we found that the smaller the number of wavelength points for each wavelength region weighting, the better the separation as was observed in the separation of 12 benign and 12 cancer lesions. For the set of 40 lesions, we obtained the best separation at p=5 wavelength points. While this is a very small range of points, it nevertheless leads to the best separation. We believe that with a significantly larger data set, the separation should be improved with longer wavelength regions for weighting, as there would be more data points to work with, and hopefully more consistency in spectral shape. Nevertheless, for this data set we systematically increased the size of the weighting wavelength regions to p=100. We found “best separation” at p=50, 100% sensitivity and 75% specificity; followed by p=70, 100% sensitivity, 72% specificity. For the clinic it is more important to have 100% sensitivity with a lower specificity value.

Once the weightings have been decided, the information will be stored into memory of the program. Then for every case which is entered as “unknown” the program should provide predictive results indicating whether the lesion is suspected to be benign or malignant. As a test of the settings and weightings defined as “best” for the set of 40 lesions, we performed a test using STC spectrum from one fibroadenoma and once cancer subject whose data has not been already input into the algorithm. Data from both patients were correctly identified. As we obtain more data we can perform more of such tests.

Finally, here we separate benign and malignant lesions using the STC spectra only. The formulas for the spectral separation and the optimization algorithm are adaptable. 1) other components such as the concentration of hemoglobin or the TOI value can be used as separation criteria 2) more than 2 lesions types can be discriminated (for example, fibroadenoma, cancers and cysts).

It can be appreciated now that we have developed an algorithm for analysis of spectra from different classification types. The algorithm calculates a wavelength weighted distance of a given spectra from the representative average spectra of each classification type. Here we show an application of the spectral separation method to STC spectra. Through this method we have demonstrated that the STC spectra can be utilized to separate fibroadenoma and cancer lesions. With this method we find that it is the spectral shape, not amplitude which can discriminate benign and malignant lesions. Once the best weighting factors have been determined, the values will be stored, and then applied to a given a spectra of “unknown” origin to determine how “distant” the spectra is from the average STC spectrum from a cancer and fibroadenoma. Separation of lesions based on spectral shapes provides new opportunities for discrimination of benign and malignant lesions using optical methods.

An Example of the Use of Optical Methods Discriminate Benign and Malignant Lesions

Through a 61 subject (22 cancer, 18 fibroadenoma, and 21 normal) study, we show that STC spectra for cancer and fibroadenoma lesions are different. Furthermore, application of the of the spectral separation method reveals that the STC spectral shape, not amplitude, can be used to discriminate benign and malignant lesions.

Materials and Methods

Patients

In a database search of patient records dating from August 2004 to January 2007, DOS data for total of 61 subjects were identified: 22 malignant tumors, 18 benign lesions, and 21 normal subjects (i.e. no known lesion). All subjects were female ranging in age from 22 to 74 years. Within each group the mean age and range was as following: 1) malignant: 47, 32-65 years; benign: 34, 22-57 years; and normal: 42, 22-74 years.

With regards to study population, baseline data obtained prior to the beginning of chemotherapy was included for patients measured under the neoadjuvant chemotherapy protocol. Data on 3 patients with malignant lesions were excluded from the final analysis due to poor quality which we suspect to be due to instrumentation issues. Data from 5 patients were excluded to maintain consistency for accurate lesion characterization: 3 patients had been injected with lymphozurin; 1 patient had silicon breast implants; and 1 patient had a retroareolar lesion. Note that one patient was measured twice during this time period as another fibroadenoma was diagnosed at a later date. All lesions studied were clinically diagnosed as cancer or fibroadenoma following confirmed biopsy results. In general, DOS measurements were obtained either before or at least 4 weeks after biopsy to avoid artifacts from bruising. For all patients, standard mammogram and ultrasound reports were obtained as part of the clinical procedures. See Tables 2-4 for patient information.

TABLE 2

| | | Menopausal |
|---|---|---|
| Age | BMI (kg/m\hat{ }2) | Status |
| 33 | NA | Pre |
| 33 | 19.22 | Pre |
| 44 | 21.25 | Pre |
| 46 | 24.74 | Post |
| 46 | 25.44 | Peri |
| 28 | 21.84 | Pre |
| 50 | 29.11 | Post |
| 59 | 24.18 | Post |
| 74 | 30.10 | Post |
| 46 | 19.46 | Pre |
| 39 | 21.99 | Pre |
| 56 | 21.99 | Post |
| 50 | 19.53 | Pre |
| 56 | 23.22 | Post |
| 22 | 18.70 | Pre |
| 24 | 20.30 | Pre |
| 26 | 36.35 | Pre |
| 45 | 17.78 | Pre |
| 59 | NA | Post |
| 31 | NA | Pre |
| 31 | 19.78 | Pre |
| 34 | 41.75 | Pre |

TABLE 3

| | BMI | Menopausal | | U/Z ACR |
|---|---|---|---|---|
| Age | (kg/m\hat{ }2) | Status | Lesion Size (mm) | Bi-Rads |
| 57 | 23.83 | Post | 12.00 × 10.00 × 6.00 | 4 |
| 38 | 29.75 | Pre | 14.00 × 13.00 × 12.00 | 4 |
| 25 | 22.91 | Pre | 22.00 × 10.00 | 3 |
| 22 | 23.68 | Pre | 35.00 × 43.00 | 4 |
| 25 | 19.69 | Pre | 7.00 × 9.00 × 4.00 | 4 |
| 40 | 20.64 | Pre | 19.00 × 8.00 × 21.00 | 2 |
| 41 | 20.95 | Pre | L: 9.00, 14.00, 7.00, 13.00, | 2 |
| | | | 9.00 | |
| 41 | 30.36 | Pre | 20.00 | 4 |
| 28 | 28.38 | Pre | L: 41.00 × 18.00 × 28.00; | 4 |
| | | | 29.00 × 12.00 × 28.00; | |
| 32 | 18.64 | Pre | 13.00 × 3.00 × 12.00 | 3 |
| 37 | 24.07 | Pre | 28.10 × 11.80; | 4 |
| | | | 13.00 | |
| 31 | 23.68 | Pre | 37.00 × 18.00 × 20.00 | 4A |
| 22 | 22.19 | Pre | 26.00 × 25.00 × 8.00 | 3 |
| 42 | 29.33 | Pre | N/Avail | No Report |
| 27 | 23.09 | Pre | 5.50 × 7.00 × 2.70 | 4A |
| 32 | 29.92 | Pre | 13.90 × 9.10 × 14.50 | 3 |
| 33 | 30.02 | Pre | 12.60 × 8.400 | No Report |

TABLE 4

| | | | | | U/Z |
|---|---|---|---|---|---|
| | BMI | Meno- | | | ACR |
| | (kg/ | pausal | Classifi- | | Bi- |
| Age | m\hat{ }2) | Status | cation | Lesion Size (mm) | Rads |
| 48 | 34.23 | Pre | IDC | 24.00 × 17.00 × 27.00 | 5 |
| 38 | 32.76 | Pre | IDC | 24.00 × 22.00 × 21.00 | 5 |
| 50 | 25.88 | Post | IDC w/ | 7.00 × 5.00 × 5.40 | 5 |
| | | | Lobular | | |
| 32 | 28.40 | Pre | IDC w/ | 16.00 × 8.00 × 18.00 | 5 |
| | | | LNmets | | |
| 53 | 26.20 | Post | IDC w/ | 32.00 × 18.00 × 12.00 | 5 |
| | | | LNmets | | |
| 47 | 22.76 | Post | IDC | 17.00 × 14.00 × 13.00 | 5 |
| 45 | 26.68 | Pre | IDC | 11.00 × 13.00 × 6.00 | 4 |
| 45 | 25.45 | Pre | IDC | 16.00 × 16.00 × 14.00 | 5 |
| 51 | 40.34 | Post | IDC | 60.00 × 27.00 | 5 |
| 36 | 24.61 | Pre | IDC | 13.00 × 9.00 × 10.00 | No |
| | | | | | Report |
| 33 | 39.79 | Pre | IDC w/ | 42.90 × 21.90 × 33.00 | 5 |
| | | | DCIS | | |
| 42 | 20.01 | Pre | IDC 80% | 26.00 × 14.00 × 10.00 | 4 |
| | | | ILC 20% | | |
| 43 | 21.66 | Pre | ILC 25% | 29.00 × 17.00 × 25.00 | 5 |
| | | | LCIS 75% | | |
| 38 | 23.74 | Pre | IDC w/ | 19.00 × 18.00 × 18.00 | No |
| | | | Focal | | Report |
| | | | DCIS | | |
| 63 | 29.30 | Post | IDC | 25.00 × 22.00 × 29.00 | 5 |
| 59 | 35.52 | Post | IDC w/ | 100.00 × 64.00 × 41.00 | 5 |
| | | | DCIS | | |
| 57 | 28.68 | Post | IDC | 31.00 × 16.00 × 19.00 | 5 |
| 45 | 26.68 | Pre | IDC | 39.00 | No |
| | | | | | Report |
| 40 | 28.38 | Pre | IDC | 34.90 × 22.70 × 25.20 | 4 |
| 55 | 27.86 | Post | IDC/ | 8.80 × 7.70 × 9.10 | 4C |
| | | | ILC | | |
| 65 | 31.31 | Post | IDC | 16.70 × 14.80 × 19.50 | 4C |
| 47 | 21.94 | Pre | IDC | 13.00 × 12.00 × 10.00 | No |
| | | | | | Report |
| 58 | 18.92 | Post | IDC | 11.00 × 7.00 × 8.00 | No |
| | | | | | Report |
| 60 | 29.27 | Post | IDC | 14.00 | 5 |

Instrumentation

DOS measurements were obtained using the Laser Breast Scanner (LBS), which is based on a diffuse optical spectroscopy technique known as steady state frequency domain photon migration (SSFDPM) as described above. In this study the LBS employs a handheld probe, containing the light source and detector fibers. The frequency domain photon migration (FDPM) component employs six laser diodes at the wavelengths of 658, 682, 785, 810, 830, and 850 nm using a multi-frequency, single source-detector separation approach. After propagation though the tissue, the intensity modulated light is detected by an avalanche photodiode detector (APD). The final output corresponds to the phase and amplitude as a function of modulation frequency. The Steady State (SS) component delivers broadband light from a high intensity tungsten-halogen lamp (Ocean Optics, Dunedin, Fla.). Changes in reflectance are measured using a spectrometer (BWTek, Newark, Del. and Oriel, Irvine, Calif.) The total acquisition time for a single measurement is near 10 seconds.

Measurement Procedure

Patients were asked to lie comfortably in a supine position. Ultrasound and pathology reports were utilized to determine type, localization, and size of lesions, thus all lesion positions were known a priori. Using a surgical pen, measurement positions were marked at 1 cm intervals in a line or grid pattern over the tumor containing tissue. Regions of interest were selected in proportion to the lesion size. The handheld probe was placed point by point over the tissue to obtain absorption and scattering spectra at each measured position. Two internal controls were measured: 1) on the opposite breast at the contralateral locations, which were assumed normal tissue unless otherwise indicated in the patient's clinical reports; 2) on the lesion breast at positions of normal tissue surrounding the tumor-containing tissue. All measurements were taken in reflection geometry, with utmost care to maintain the probe in gentile contact with the surface skin without compression.

Data Analysis

Data were analyzed using custom made software using the MATLAB program. For each measurement position on the breast, the data corresponding to the phase and amplitude were obtained from the FDPM and reflectance spectra from the SS underwent processing to recover the scatter-corrected absorption spectra for the full bandwidth of 650-1000 nm. The absorption spectra were then further analyzed by the custom designed Elantest software to obtain the Specific tissue component (STC) absorption spectra using the double-differential method of near-infrared spectral analysis.

Details of the double-differential method have been outlined above. Briefly, in the double-differential method the goal is determine if there are other spectral differences that cannot be accounted for by the different amounts of the four basis components (oxyhemoglobin, deoxyhemoglobin, bulk lipid and water). First the average spectrum of the normal breast tissue is calculated. Then the difference between this average spectrum and the spectrum at each location (including the normal breast) is determined. If the only components present are the ones included in the four basis spectra, then the difference spectrum (at each location) can be completely fitted by the using the four components. However, if the fit is not perfect, the residual of the fit will provide the additional spectra which are not included in the four basis components. This residual is the Specific tissue component (STC).

STC absorption spectra for all lesions were analyzed over a line scan of points over the lesion-containing tissue including the surrounding normal tissue. (A line scan is defined as a line of points which extends from the normal tissue on one side of the lesion, over the lesion, and to the other side of the lesion.) Spectra were analyzed over the lesion-containing tissue (for a total of 40 lesions, 22 malignant and 18 benign) as well as at the equivalent region of normal tissue on the contralateral side. Furthermore, data from a group of 21 normal subjects, with no known lesion, were studied as an additional control. In order to analyze the data consistently, regions on normal subjects were randomly chosen to “represent lesion” thereby obtaining the equivalent analysis set-up with lesion and normal breast, tumor-containing and normal tissue. STC index were obtained and plotted to determine spatial localization and extent of STC spectral features. The STC index is a quantitative measure of the STC by summing the variation in specific wavelength regions found to show differences in comparison to normal tissue (650-665 nm, 730-800 nm, 930-960 nm, and 980-1000 nm). Details have been outlined elsewhere

STC spectra for malignant and benign lesions were further explored for differential diagnosis by applying a spectral separation method. The details of which are described above. In brief, the developed algorithm maximizes for differences in spectral shape by weighting different wavelength regions. For every patient, (for each spectrum) the “difference” from the average STC spectrum of a fibroadenoma and a cancer are calculated and plotted on a x-y coordinate system. The position of a given spectra on the map is indicative of the similarity to the average STC spectrum for fibroadenoma and cancer.

Statistical Analysis

Sensitivity, specificity, positive predictive value, and negative predictive value were calculated as defined: 1) sensitivity: TP/(TP+FN); 2) specificity: TN/(TN+FP); 3) positive predictive value: TP/(TP+FP); and 4) negative predictive value: TN/(TN+FN).

Results

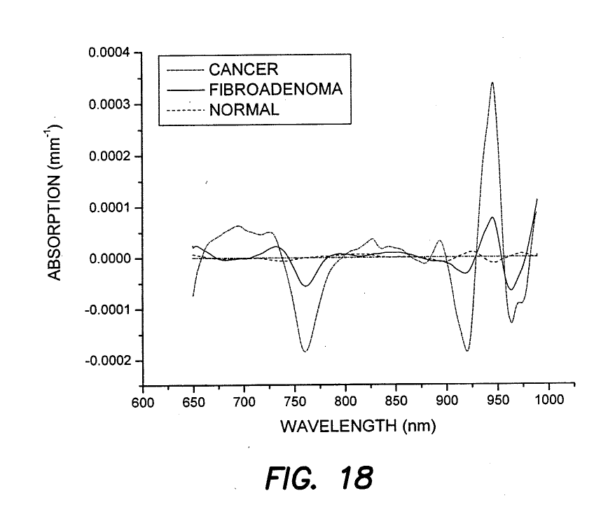

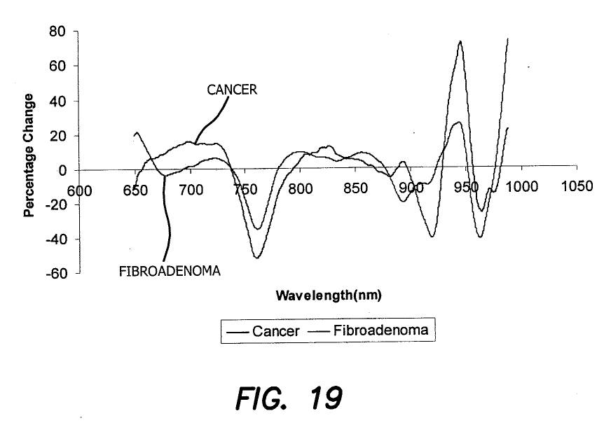

We obtained STC spectra from DOS measurements on tissue from normal, benign, and malignant cases. Spectra were calculated using the algorithm for the double-differential approach to spectral analysis as described above. In FIG. 18 we show the STC spectra averaged over a line scan of points for normal, benign, and malignant cases. As mentioned earlier, a complete “line scan” of data extends from normal tissue on one side of the lesion, over the lesion, and to the other side. In the 21 normal subjects measured, STC spectra revealed to be essentially flat and featureless. Similarly, the STC absorption spectra from normal tissue averaged for all 40 lesion patients displayed a featureless line across the wavelengths. In FIG. 18 we show the average STC spectra for the 61 “normal tissue” positions. In contrast STC spectra for malignant and benign lesions were not featureless, each type of lesion revealed a different shape with distinctive peaks across the wavelength region of 650-1000 nm. In FIG. 19 we show the STC spectra for the 18 fibroadenoma and 22 cancer lesions after amplitude normalization. Compared to the STC absorption spectra over normal tissue, these lesion spectra exhibit roughly 4 regions where notable differences were observed: 650-730 nm, 730-800 nm, 930-980 nm, 980-1000 nm.

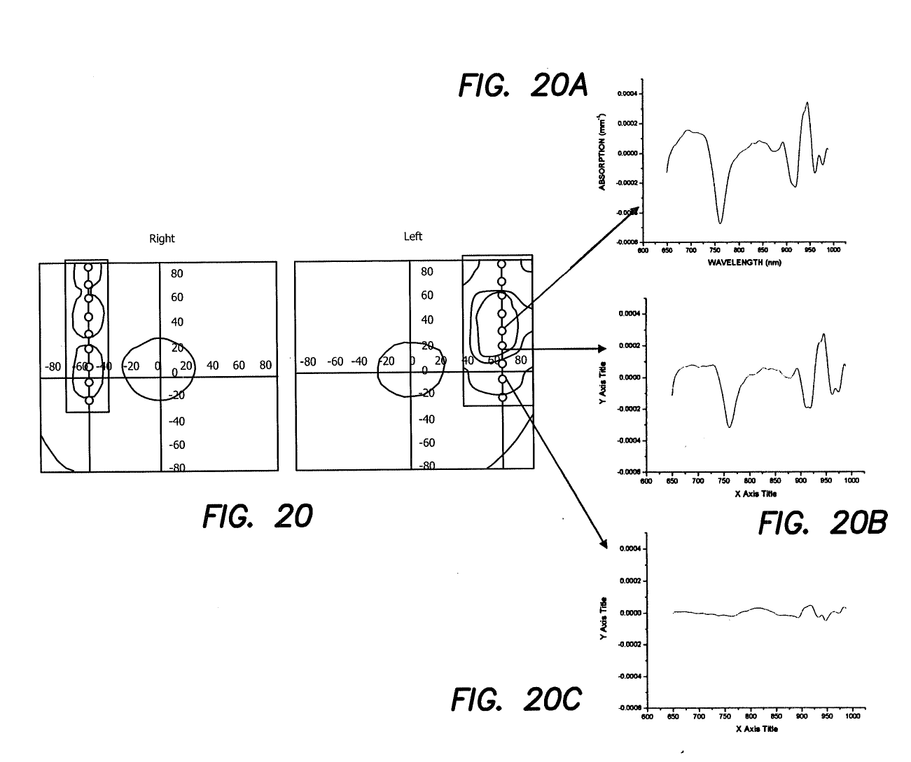

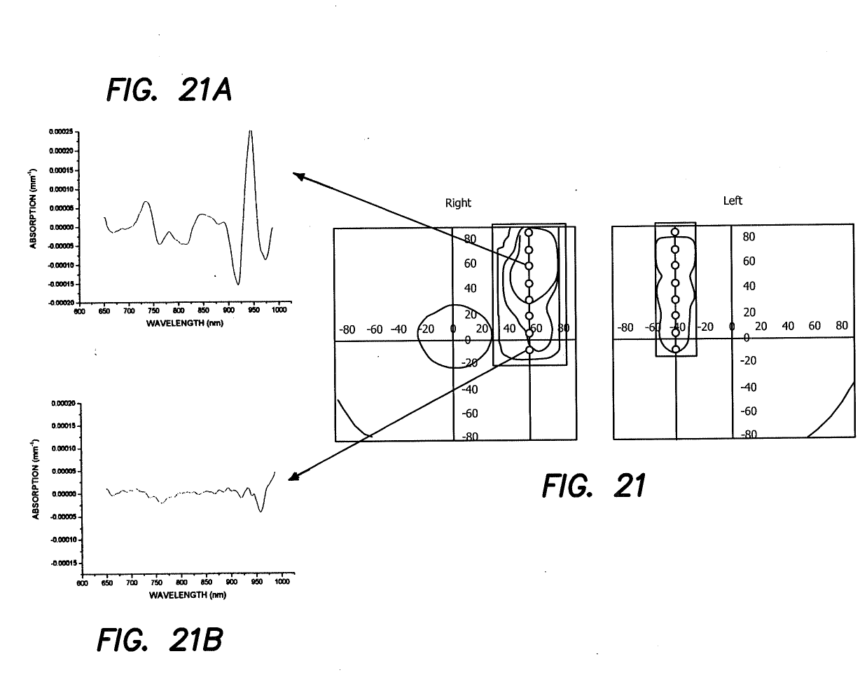

The STC spectra exhibiting tumor are spatially localized to the regions of tumor-containing tissue. In FIG. 20 and FIG. 21 we show an example of the spatial extent of the STC absorption spectra for a fibroadenoma and cancer lesion. The images of the STC analysis have been obtained by using the STC Index. Note that the STC spectra for the lesion are only found in the lesion-containing location as noted from the ultrasound reports.

In FIG. 20 we show the STC index-based image for a cancer lesion measuring 11 mm by 13 mm. This data was obtained for a 45 year old woman who was pre/post menopausal diagnosed with invasive ductal carcinoma cancer in the left/right breast. Note that the STC absorption spectra over the lesion-containing region are different than those over the surrounding normal tissue.

In FIG. 21 we show the STC absorption spectra for a 22 year old pre/post menopausal woman with 35 mm by 43 mm fibroadenoma in the right/left breast. Again we note that the STC spectra over the lesion is characteristic of a fibroadenoma whereas over normal tissue the STC absorption spectra is relatively featureless.

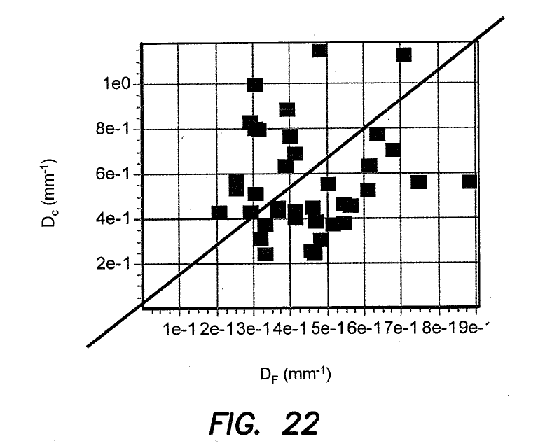

We were able to discriminate between benign and malignant lesions using the STC absorption spectra using feature analysis. The difference between a given spectrum (from every lesion patient) from the average spectrum of each type of lesion were quantified. A map of the “similarity” to each lesion-type were plotted in FIG. 22. The x axis represents the “similarity” of a given spectrum from the average STC spectrum of a fibroadenoma, and the y axis represents the “similarity” of a given spectrum from the average STC spectrum of cancer. The units are in absorption (mm⁻¹). Using the spectral separation method malignant and benign lesions were separated using the STC spectra resulting in a sensitivity of 100% (22 of 22 lesions), and specificity of 92% (22 of 24 lesions). Positive predictive values and negative predictive values were calculates to be 92% (22 of 24 lesions) and 100% (18 of 18 lesions), respectively. Two benign lesions were misclassified as malignant. Lesions were separated by drawing a line of unity slope through the origin as shown on in FIG. 22.

The double-differential approach to spectral analysis reveals STC spectra, specific absorption bands present only in tumor-containing regions, and not in normal tissue. These absorption bands are due to subtle spectral shifts which arise after accounting for subject-specific physiological variation as well as absorption due to the major breast tissue absorbing components in near-infrared, namely hemoglobin, bulk lipid and water. In this study our goal was to identify the STC spectra of benign and malignant lesions were different, and determine whether they could be used for discrimination. Our results suggest that the STC absorption bands are lesion-type specific. Furthermore, these STC spectra can be used for differential diagnosis. Discrimination of lesion type was obtained by spectral shape, the amplitude (or amount of the STC absorption spectrum) is not a necessary discriminating parameter.

Note that the double-differential method is different from the conventional near-infrared spectral analysis approach whereby tumors are separated from normal or benign tissues by thresholding tissue parameters including oxyhemoglobin, deoxyhemoglobin, and oxygen saturation from the background. Furthermore, unlike many other groups we do not perform a tomographic reconstruction, nor do we use any spatial “priors” such localization information from ultrasound or x-ray to create an image.

While the amplitudes of the STC spectra are small (about 1% of the original spectra), the spectra are highly reproducible and have a high signal to noise ratio, having wavelength dependent characteristics which are lesion-type specific as seen from this study of 40 lesions. Although both amplitude and spectral shape can be used for separating benign and malignant lesions, we found that spectral shape alone is sufficient. For this data set the best separation was obtained after normalizing the amplitudes, followed by weighting in order to bring out the differences.

We find that the shape of the STC spectra is conserved regardless of size of lesion. In this study STC absorption signatures were identified for a range of lesions sizes from 7-43 mm in largest dimension. The depth of the lesion was not available. We hypothesize that the lesion depth may affect amplitude of STC spectra, but will not affect spectral shape. This conservation of STC shape regardless of lesion size and depth is an important advancement in optical spectroscopy as other methods of analysis rely on thresholding of parameters (such as hemoglobin) to classify the lesion as normal, benign or malignant. For these methods there is a sampling issue, as the smaller or deeper the lesion, the more “normal” tissue is measured as opposed to lesion tissue. This can lead to smaller values of the parameter leading to misclassification.

There were several limitations of the study, some of which can be addressed by a larger clinical trial. To begin with fibroadenoma were the only type of benign lesions measured. For a more complete analysis, other types of benign lesions should be measured such as cysts or fibrocystic changes. In this study the goal was to characterize the STC spectra for malignant and benign lesions. Thus we did not include any cases where absorption from artifacts may confound results: 3 patients administered with lymphozurin, which is absorbing in the 650-1000 nm wavelength, were excluded as the lymphozurin was only injected in the lesion breast. The double differential approach relies on the presence of comparatively “normal” tissue for a given patient to serve as the internal control. Thus if lymphozurin had been accounted for in “normal” tissue, theoretically it could be subtracted.

Furthermore the method is adaptable in that the normal tissue need not be from the contra-lateral breast. Normal tissue from the lesion side can be used as was done in the analysis of one patient for whom data was not available from the contra lateral side, and for one patient who had bilateral lesions.

One of the limitations of optical methods has been in imaging of lesions in the region of areolar complex. Since the tissue is generally dark in this area, light perturbations due to absorption by this region may be confounding to the effects of light absorption by lesions. Given that the double-differential method relies on the differences in tissue regions, the effect of the areola can be corrected by referencing the areola region for the normal contra-lateral breast. Similarly the effect of breast implants could be accounted for. In this study we excluded one patient with a retroareolar lesion and one with breast implants as there was not enough data to make definitive conclusions with only one patient in each category.

Two fibroadenomas were misclassified as malignant lesions. One of the missed lesions was 35×43 mm, while the other one was very small, 9×7 mm. While the lesions were misclassified, both lesions were identified as lesions by the STC spectra. According to the “similarity” map of FIG. 22, the STC of these lesions appear to be more similar to the average STC spectrum of the cancer lesions as opposed to fibroadenomas. In this study we were interested in the separation of benign and malignant lesions using only the STC spectrum. Additional functional characterization from other optical parameters such as concentrations of hemoglobin, bulk lipid and water may provide more insight for improved separation.

We envision DOS to potentially serve as an adjunct to conventional breast screening techniques. Once an anomalous mass has been identified by standard clinical procedures, DOS can provide functional characterization of the tissue volume non-invasively and provide results immediately at the time of examination. DOS measurements must be obtained over the suspicious region as well as over tissue known to be normal (to serve as a comparison). Automatically the computer software should run an analysis and display the location of the lesion on the map as presented in FIG. 22. Position on the map is indicative of how similar the STC spectra of the unknown lesion is to the average STC spectra of a fibroadenoma and the average STC spectra of a cancer as determined from a large database of lesions. We expect the addition of functional characterization to facilitate medical diagnosis.

In conclusion the double-differential method relies on the presence or absence of an absorption band signature, and not the amount of a certain component (for example the hemoglobin concentration). These changes arise due to subtle spectral shifts as the changes in the bulk tissue properties (oxyhemoglobin, deoxyhemoglobin, bulk lipid and water) as well as the individual physiological variation have been accounted for. Application of this method has shown STC (Specific tissue component) absorption bands to be specific in two very important ways: localization and pathology type. These signatures are spatially localized to the tumor containing regions of the breast. Furthermore, results from this pilot study of 61 subjects (21 normal and 40 lesions) have shown that normal tissue result in featureless STC spectra, while the cancer and fibroadenoma lesions each exhibit different spectroscopic absorption signatures. The shape of these STC spectra can be used for discrimination of benign and malignant lesions. The observation of specific tumor spectral signatures provides new possibilities for the application of optical methods for functional characterization of tissue non-invasively.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. An improvement in a method of optically analyzing tissue in vivo in an individual to obtain a unique spectrum for the tissue of the individual, the improvement comprising:
optically measuring the tissue of the individual to obtain a spectrum of an optical parameter; and
identifying a spectral signature specific to a metabolic or physiologic state in the tissue of the individual with a unique spectrum for the tissue by considering only the spectral differences between a first metabolic or physiologic state of the tissue of the individual and one or more other metabolic or physiologic states of the tissue of the individual such that identification of the spectral signature is self-referencing with respect to intra-individual metabolic or physiologic variations.

2. The improvement of claim 1 where identifying the spectral signature specific to the metabolic or physiologic state in the tissue of the individual comprises:
subtracting the absorption spectrum of the first metabolic or physiologic state of the tissue from absorption spectrum obtained at different locations on tissue having at least one of the other metabolic or physiologic states to obtain a difference spectrum;
fitting the difference spectrum to spectral basis components; and
analyzing residuals of the spectral basis components from the fitted difference spectrum.

3. The improvement of claim 1 where identifying the spectral signature specific to the metabolic or physiologic state in the tissue of the individual comprises obtaining a complete absorption spectrum of the tissue across the full IR, near-IR, or visible wavelength range.

4. The improvement of claim 1 where identifying the spectral signature specific to the metabolic or physiologic state in the tissue of the individual comprises separating out a scattering spectrum and obtaining an absolute absorption spectrum.

5. The improvement of claim 1 where identifying the spectral signature specific to the metabolic or physiologic state in the tissue of the individual comprises analyzing a near infrared spectrum of regions of a breast with a tumor by comparison between regions of normal breast tissue and tumor breast tissue by first subtracting the spectrum of the normal breast tissue of the individual from the spectrum obtained at different locations in the breast tissue with the tumor of the individual to obtain a differential spectrum, then fitting the differential spectrum using a basis component spectrum to obtain a fitted spectrum, and analyzing residues of the fitted spectrum.

6. The improvement of claim 1 where identifying the spectral signature specific to the metabolic or physiologic state in the tissue of the individual comprises identifying intrinsic spectroscopic markers of the tissue in the near-IR.

7. The improvement of claim 6 where identifying intrinsic spectroscopic markers of the tumor in the near-IR comprises identifying characteristic absorption bands indicative of state changes related to lipid, water, hemoglobin, derivatives of hemoglobin, or an optical absorber.

8. The improvement of claim 7 where identifying characteristic absorption bands in the lipid region comprises characterizing variations in a water band in the 980 nm region in a tumor region of the individual compared to the normal tissue of the individual.

9. The improvement of claim 1 where identifying the spectral signature specific to the metabolic or physiologic state in the tissue of the individual comprises combining information relating to spectral differences between tissue of the individual characterized by the first metabolic or physiologic state and tissue of the individual characterized by one or more other metabolic or physiologic states to construct an index that is characteristic of a region characterized by the one or more other metabolic or physiologic states on the basis of the tissue composition and/or molecular disposition of tissue components.

10. The improvement of claim 1 where identifying the spectral signature specific to the metabolic or physiologic state in the tissue of the individual comprises automatically identifying a spectral signature specific to the one or more other metabolic or physiologic states by a computer algorithmic procedure without physician intervention.

11. An improvement in an apparatus for analyzing tissue composition in vivo in a individual to obtain a unique spectrum for the tissue of the individual, the improvement comprising:
means for optically measuring the tissue of the individual to obtain a spectrum of an optical parameter; and
means for identifying a spectral signature specific to a metabolic or physiologic state in the tissue of the individual with a unique spectrum for the tissue by considering only the spectral differences between a first metabolic or physiologic state of the tissue of the individual and one or more other metabolic or physiologic states of the tissue of the individual such that identification of the spectral signature is self-referencing with respect to intra-individual metabolic or physiologic variations.

12. The improvement of claim 11 where the means for identifying a spectral signature specific to a metabolic or physiologic state in the tissue of the individual comprises:
means for subtracting the absorption spectrum of the first metabolic or physiologic state of the tissue from absorption spectrum obtained at different locations on tissue having at least one of the other metabolic or physiologic states to obtain a difference spectrum;
means for fitting the difference spectrum to spectral basis components; and
means for analyzing residuals of the spectral basis components from the fitted difference spectrum.

13. The improvement of claim 11 where means for identifying the spectral signature specific to the metabolic or physiologic state in the tissue of the individual comprises means for obtaining a complete absorption spectrum of the tissue across the full IR, near-IR, or visible wavelength range.

14. The improvement of claim 11 where the means for identifying the spectral signature specific to the metabolic or physiologic state in the tissue of the individual comprises means for separating out a scattering spectrum and means for obtaining an absolute absorption spectrum.

15. The improvement of claim 11 where the means for identifying the spectral signature specific to the metabolic or physiologic state in the tissue of the individual comprises means for analyzing a near infrared spectrum of regions of a breast with a tumor by comparison between regions of normal breast tissue and tumor breast tissue by first subtracting the spectrum of the normal breast tissue of the individual from the spectrum obtained at different locations in the breast tissue with the tumor of the individual to obtain a differential spectrum, means for then fitting the differential spectrum using a basis component spectrum to obtain a fitted spectrum, and means for analyzing residues of the fitted spectrum.

16. The improvement of claim 11 where the means for identifying the spectral signature specific to the metabolic or physiologic state in the tissue of the individual comprises means for identifying intrinsic spectroscopic markers for a tumor of the tissue in the near-IR.

17. The improvement of claim 16 where the means for identifying intrinsic spectroscopic markers of the tumor in the near-IR comprises means for identifying characteristic absorption bands indicative of state changes related to lipid, water, hemoglobin, derivatives of hemoglobin, or an optical absorber.

18. The improvement of claim 17 where the means for identifying characteristic absorption bands in the lipid region comprises means for characterizing variations in a water band in the 980 nm region in a tumor region of the individual compared to the normal tissue of the individual.

19. The improvement of claim 11 where the means for identifying the spectral signature specific to the metabolic or physiologic state in the tissue of the individual comprises means for combining information relating to spectral differences between tissue of the individual characterized by the first metabolic or physiologic state and tissue of the individual characterized by one or more other metabolic or physiologic states to construct an index that is characteristic of a region characterized by the one or more other metabolic or physiologic states on the basis of the lipid composition and/or bound water.

20. The improvement of claim 11 where the means for identifying the spectral signature specific to the metabolic or physiologic state in the tissue of the individual comprises means for automatically identifying a spectral signature specific to the one or more other metabolic or physiologic states by a computer algorithmic procedure without physician intervention.

21. A software program recorded on a medium containing instructions for controlling a measurement and computer system for performing the improvement in the method of claim 1.

22. The improvement of claim 1 where identifying the spectral signature specific to a metabolic or physiologic state in the tissue of the individual with a unique spectrum for the tissue comprises separating tissue having the first metabolic or physiologic state from tissue having the one or more other metabolic or physiologic states using only one or more characteristics of shape of the spectrum.

23. The improvement of claim 22 where separating tissue having the first metabolic or physiologic state from tissue having the one or more other metabolic or physiologic states using only one or more separation characteristics of shape of the spectrum comprises separating benign and malignant lesions using only spectral shape.

24. The improvement of claim 22 where separating tissue having the first metabolic or physiologic state from tissue having the one or more other metabolic or physiologic states using only one or more separation characteristics of shape of the spectrum comprises using concentration of hemoglobin or tissue optical index (TOI) value as a separation characteristic.

25. The improvement of claim 23 where separating tissue having the first metabolic or physiologic state from tissue having the one or more other metabolic or physiologic states using only one or more separation characteristics of shape of the spectrum comprises discriminating more than two lesions types.

26. The improvement of claim 22 where separating tissue having the first metabolic or physiologic state from tissue having the one or more other metabolic or physiologic states using only one or more characteristics of shape of the spectrum comprises determining a wavelength weighted distance of a given specific tissue component (STC) spectrum from a representative average spectrum of each metabolic or physiologic state of tissue.

27. The improvement of claim 22 where separating tissue having the first metabolic or physiologic state from tissue having the one or more other metabolic or physiologic states using only one or more characteristics of shape of the spectrum comprises computationally determining spectral shape to discriminate between each metabolic or physiologic state of tissue.

28. The improvement of claim 22 where separating tissue having the first metabolic or physiologic state from tissue having the one or more other metabolic or physiologic states using only one or more characteristics of shape of the spectrum comprises computationally determining spectral shape to discriminate between benign and malignant lesions.

29. The improvement of claim 26 where determining a wavelength weighted distance of a given spectrum from the representative average spectrum comprises determining a best set of weighting factors, storing the weighting factors, applying the stored weighting factors to a given a spectrum of unknown origin, and determining how distant the spectrum is from the average STC spectrum for each metabolic or physiologic state of tissue.

30. The improvement of claim 1 where optically measuring the tissue of the individual to obtain a spectrum of an optical parameter comprises obtaining the spectral signature as an absorption spectrum by combining frequency-domain and steady state measurements, by performing steady state measurements only, by performing time-domain measurements, or by performing spatially resolved measurements.
